(19) United States
(12) Patent Application Publication
(10) Pub. No.: US 2022/0033912 A1
(43) Pub. Date: Feb. 3, 2022

(54) TRANSCRIPTOMIC PROFILING FOR PROGNOSIS OF BREAST CANCER TO IDENTIFY SUBJECTS WHO MAY BE SPARED ADJUVANT SYSTEMIC THERAPY

(71) Applicants: PFS Genomics Inc, Vancouver, CA (US); DECIPHER BIOSCIENCES, INC., San Diego, CA (US)

(72) Inventors: S. Laura Chang, Ann Arbor, MI (US); Elai Davicioni, La Jolla, CA (US); Felix Feng, Hillsborough, CA (US)

(21) Appl. No.: 17/311,995

(22) PCT Filed: Dec. 6, 2019
(86) PCT No.: PCT/US2019/065098

§ 371 Date: Jun. 8, 2021

Related U.S. Application Data
(60) Provisional application No. 62/777,128, filed on Dec. 8, 2018.

(51) Int. Cl. C12Q 1/6886; C12Q 1/6816; G01N 33/574; G16H 50/20

(52) U.S. Cl. CPC C12Q 1/6886; G16H 50/20; G01N 33/574; C12Q 1/6816

(57) ABSTRACT

Methods, systems, and kits for the diagnosis, prognosis, and treatment of breast cancer in a subject are disclosed. The invention also provides biomarkers and clinically useful classifiers for identifying subjects at low risk of breast cancer recurrence who may be spared from adjuvant chemotherapy and endocrine therapy. Further disclosed herein, in certain instances, are probe sets for use in detecting such biomarkers for determining the risk of breast cancer recurrence in a subject. Methods of treating breast cancer based on expression profiling to determine the risk of breast cancer recurrence are also provided.

### RELATED APPLICATIONS

This application is a 35 USC § 371 National Stage application of International Application No. PCT/US2019/065098 filed Dec. 6, 2019, now pending; which claims the benefit under 35 USC § 119(e) to U.S. Provisional Patent Application Ser. No. 62/777,128, filed on Dec. 8, 2018 now expired. The disclosure of each of the prior applications is considered part of and is incorporated by reference in the disclosure of this application.

### FIELD OF THE INVENTION

The present invention pertains to the field of personalized medicine and methods for treating breast cancer. In particular, the invention relates to the use of transcriptomic profiling for prognosis of breast cancer to identify subjects who may be spared adjuvant systemic therapy.

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, breast cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if breast cancer cells spread to a bone (or anywhere else), it can mean that the individual has metastatic breast cancer.

Standard clinical parameters such as tumor size, grade, lymph node involvement and tumor-node-metastasis (TNM) staging (American Joint Committee on Cancer) may correlate with outcome and serve to stratify subjects with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual subject care and treatment.

The treatment of primary breast cancer is highly individualized and has entered the era of precision medicine. Due to increased public awareness and intensified screening programs, the proportion of low-risk tumors has increased with a corresponding risk of over-treatment (Hosseini et al. (2017) Eur. J. Surg. Oncol. 43:938-943). Thus, current guidelines focus on de-escalating treatment in low-risk subjects, in addition to escalating treatment of high-risk tumors (Curigliano et al. (2017) Ann Oncol 28:1700-1712). Tools to help assess recurrence risk reduction with adjuvant chemotherapy have been the most successful and incorporated into clinical guidelines, while no tests are currently approved to stratify subjects by benefit from endocrine therapy except the expression of the estrogen receptor (ER) (Curigliano et al., supra).

A large meta-analysis from the Early Breast Cancer Trialists' Collaborative Group (EBCTCG) confirmed that ER-positive (ER+) breast cancer continues to recur and cause death at a relatively consistent rate over 15 years after stopping endocrine therapy, suggesting the need for prolonged endocrine therapy in certain subjects (Pan et al. (2017) N. Engl. J. Med. 377:1836-1846). Endocrine therapy, however, may have substantial side-effects, which is reflected in an adherence rate of 50-80% (Chlebowski et al. (2006) Oncology 71:1-9). This has led to efforts in identifying subjects with ER+ cancers in whom endocrine therapy can be safely omitted. Thus, a test which could select those women at lowest risk for recurrence even in the absence of endocrine therapy could spare unnecessary treatment and its associated morbidities.

To address this, the 70-gene signature was recently shown to identify an ultra-low risk group of subjects with low risk of recurrence over 20 years, even without any systemic treatment (Esserman et al. (2017) JAMA Oncol. 3:1503-1510). Interestingly, the same authors evaluated the effect of giving tamoxifen to high- and low-risk subjects, as stratified by the 70-gene signature, and found that adjuvant tamoxifen had a significant effect in both groups (Van't Veer et al. (2017) Breast Cancer Res Treat 166:593-601). This confirms that prognostication does not lead to treatment prediction, and caution should be applied when performing retrospective analysis of treatment effect in subgroups.

From a clinical perspective, it has been argued that one could consider the relative treatment effect to be consistent over subgroups, and make treatment decisions based on baseline risk level and absolute treatment effect (Peto et al. (2011) Br. J. Cancer 104:1057-1058). However, when considering use of baseline risk for gene expression tests, an emerging problem is the notion that current tests are discordant at the individual subject level. Although gene expression tests in general perform well, with similar performance on the group level, there may be substantial discordance in results for an individual subject, i.e. the low-risk and high-risk subject groups are not the same when using different tests. Indeed, a recent study found the agreement of five common gene expression tests to be only modest, with 39% of subjects classified uniformly as low-risk by all tests, while the individual tests predicted 61%-82% to be low-risk (Bartlett et al. (2016) Comparing Breast Cancer Multiparameter Tests in the OPTIMA Prelim Trial: No Test Is More Equal Than the Others. J. Natl. Cancer Inst. 108, 2016). Therefore, there remains a need for better methods to stratify subjects with early breast cancer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to methods, systems, and kits for the diagnosis, prognosis, and treatment of breast cancer in a subject. The invention also provides biomarkers and classifiers for identifying subjects at low risk of breast cancer recurrence who may be spared from adjuvant chemotherapy and endocrine therapy. Further disclosed herein, in certain instances, are probe sets for use in detecting such biomarkers for determining the risk of breast cancer recurrence in a subject. Methods of treating breast cancer based on expression profiling to determine the risk of breast cancer recurrence are also provided.

In some embodiments, the invention includes a method for determining a prognosis and treating a subject for breast cancer, the method comprising: a) obtaining a biological sample comprising breast cancer cells from the subject; b) measuring expression levels of a plurality of genes selected from Table 2 in the biological sample; c) determining if the subject is at low risk of cancer recurrence based on the expression levels of the plurality of genes selected from Table 2 in the biological sample; and d) administering adjuvant chemotherapy or endocrine therapy if the subject is not identified as being at low risk of cancer recurrence, and not administering adjuvant chemotherapy or endocrine therapy if the subject is identified as being at low risk of cancer recurrence based on the expression levels of the plurality of genes selected from Table 2 in the biological sample. In other embodiments, the levels of expression of one or more of the genes selected from Table 2 may be increased or decreased compared to a control. In yet other embodiment, the expression levels of all of the genes selected from Table 2 are measured in the biological sample.

In some embodiments, the plurality of genes are selected from the group consisting of ATP binding cassette subfamily F member 1 (ABCF1), acetyl-CoA acetyltransferase 2 (ACAT2), actin like 6A (ACTL6A), apolipoprotein B mRNA editing enzyme catalytic subunit 3B (APOBEC3B), anti-silencing function 1B histone chaperone (ASFIB), ATP synthase, H+ transporting, mitochondrial F1 complex, gamma polypeptide 1 (ATP5C1), bystin like (BYSL), chromosome 1 open reading frame 106 (C1orf106), cell division cycle 25B (CDC25B), cell division cycle 45 (CDC45), chromatin licensing and DNA replication factor 1 (CDT1), CCAAT/enhancer binding protein gamma (CEBPG), centromere protein I (CENPI), chromatin assembly factor 1 subunit A (CHAF1A), chromatin assembly factor 1 subunit B (CHAFIB), checkpoint kinase 1 (CHEKI), cytokine induced apoptosis inhibitor 1 (CIAPIN1), cytoskeleton associated protein 2 (CKAP2), CTP synthase 1 (CTPS1), DNA replication helicase/nuclease 2 (DNA2), DNA methyltransferase 1 (DNMT1), DNA methyltransferase 3 beta (DNMT3B), E2F transcription factor 8 (E2F8), emopamil binding protein (sterol isomerase) (EBP), eukaryotic translation initiation factor 4E binding protein 1 (EIF4EBP1), ER membrane protein complex subunit 8 (EMC8), family with sequence similarity 96 member B (FAM96B), Fanconi anemia complementation group A (FANCA), F-box protein 5 (FBXO5), glyceraldehyde-3-phosphate dehydrogenase (GAPDH), glycyl-tRNA synthetase (GARS), geminin, DNA replication inhibitor (GMNN), G-protein signaling modulator 2 (GPSM2), GTP binding protein 4 (GTPBP4), hepatoma-derived growth factor (HDGF), hematological and neurological expressed 1 (HN1), heat shock protein family A (Hsp70) member 14 (HSPA14), heat shock protein family D (Hsp60) member 1 (HSPD1), inositol monophosphatase 2 (IMPA2), interleukin 1 receptor associated kinase 1 (IRAKI), lysyl-tRNA synthetase (KARS), kinetochore associated 1 (KNTC1), LDL receptor related protein 8 (LRP8), minichromosome maintenance complex component 5 (MCM5), minichromosome maintenance complex component 7 (MCM7), MIS18 kinetochore protein A (MIS18A), mitochondrial ribosomal protein L15 (MRPL15), mitochondrial ribosomal protein S17 (MRPS17), mitochondrial transcription termination factor 3 (MTERF3), nuclear prelamin A recognition factor (NARF), non-SMC condensin I complex subunit D2 (NCAPD2), non-SMC condensin II complex subunit D3 (NCAPD3), NADH:ubiquinone oxidoreductase subunit A9 (NDUFA9), nuclear envelope integral membrane protein 1 (NEMP1), NME/NM23 nucleoside diphosphate kinase 1 (NME1), NOP2 nucleolar protein (NOP2), nucleoporin 205 (NUP205), origin recognition complex subunit 1 (ORC1), pyruvate dehydrogenase (lipoamide) alpha 1 (PDHA1), prenyl (decaprenyl) diphosphate synthase, subunit 1 (PDSS1), phosphoglycerate kinase 1 (PGK1), polo like kinase 4 (PLK4), purine nucleoside phosphorylase (PNP), DNA polymerase alpha 2, accessory subunit (POLA2), protein phosphatase, Mg2+/Mn2+ dependent 1G (PPM1G), peroxiredoxin 4 (PRDX4), proteasome subunit beta 4 (PSMB4), proteasome subunit beta 5 (PSMB5), proteasome 26S subunit, non-ATPase 14 (PSMD14), proteasome 26S subunit, non-ATPase 2 (PSMD2), proteasome assembly chaperone 1 (PSMG1), phosphatidylserine synthase 1 (PTDSS1), RAD51 recombinase (RAD51), RAD54 homolog B (S. cerevisiae) (RAD54B), RAD54-like (S. cerevisiae) (RAD54L), RAN binding protein 1 (RANBP1), small nuclear ribonucleoprotein polypeptide A (SNRPA1), small nuclear ribonucleoprotein polypeptide G (SNRPG), SPC25, NDC80 kinetochore complex component (SPC25), SRSF protein kinase 1 (SRPK1), stress induced phosphoprotein 1 (STIP1), transforming acidic coiled-coil containing protein 3 (TACC3), transcription elongation factor B subunit 1 (TCEB1), transmembrane protein 97 (TMEM97), topoisomerase (DNA) II alpha (TOP2A), topoisomerase (DNA) II binding protein 1 (TOPBP1), triosephosphate isomerase 1 (TPI1), uracil DNA glycosylase (UNG), and tyrosine 3-monooxygenase/tryptophan 5-monooxygenase activation protein zeta (YWHAZ).

In certain embodiments, the subject has estrogen receptor positive (ER+), human epidermal growth factor receptor 2 negative (HER2−) breast cancer and is post-menopausal.

The biological sample obtained from a subject is typically a breast biopsy or tumor sample, but can be any sample from bodily fluids or tissue of the subject that contains breast cancer cells. In certain embodiments, nucleic acids (e.g., RNA transcripts) comprising sequences from genes selected from Table 2, or complements thereof, are further isolated from the biological sample, and/or purified, and/or amplified prior to analysis.

The expression levels of biomarkers can be determined by a variety of methods including, but not limited to in situ hybridization, PCR-based methods, array-based methods, immunohistochemical methods, RNA assay methods, and immunoassay methods. Levels of gene expression can be determined, for example, using one or more reagents such as nucleic acid probes, nucleic acid primers, and antibodies. In certain embodiments, determining the level of expression of a biomarker comprises measuring the level of a nucleic acid (e.g., RNA transcript).

In some embodiments, the level of expression of at least one gene is reduced compared to a control. In other embodiments, the level of expression of at least one gene is increased compared to a control.

In some embodiments, the methods described herein are performed prior to treatment of the subject with adjuvant chemotherapy or endocrine therapy.

In other embodiments, the method further comprises calculating an average genomic risk (AGR) score for the subject, wherein adjuvant chemotherapy or endocrine therapy is administered to the subject if the subject is not identified as being at low risk of cancer recurrence based on both the AGR score and the expression levels of the genes selected from Table 2 in the biological sample, and adjuvant chemotherapy or endocrine therapy is not administered to the subject if the subject is identified as being at low risk of cancer recurrence based on both the AGR score and the expression levels of the genes selected from Table 2 in the biological sample.

In some embodiments, the significance of the expression levels of one or more biomarker genes may be evaluated using, for example, a T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). In other embodiments, the significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) or Multivariable Analysis Hazard Ratio P-value (mvaHRPval).

In yet other embodiments, the invention includes a probe set for determining a prognosis of a subject having breast cancer and whether or not to treat the subject with adjuvant chemotherapy and endocrine therapy, the probe set comprising a plurality of probes for detecting a plurality of target nucleic acids, wherein the plurality of target nucleic acids comprises one or more gene sequences, or complements thereof, of genes selected from Table 2. Probes may be detectably labeled to facilitate detection.

In other embodiments, the invention includes a system for determining a prognosis of a subject who has breast cancer and whether or not to treat the subject with adjuvant chemotherapy and endocrine therapy, the system comprising: a) a probe set described herein; and b) a computer model or algorithm for analyzing an expression level or expression profile of the plurality of target nucleic acids hybridized to the plurality of probes in a biological sample from a subject who has breast cancer and determining if the subject is at low risk of cancer recurrence based on the expression level or expression profile and can be spared treatment with adjuvant chemotherapy and endocrine therapy.

In some embodiments, the invention includes a kit for determining a prognosis of a subject having breast cancer and whether or not to treat the subject with adjuvant chemotherapy and endocrine therapy, the kit comprising agents for measuring levels of expression of a plurality of genes selected from Table 2. In other embodiments, the kit may include one or more agents (e.g., hybridization probes, PCR primers, or microarray) for measuring levels of expression of a plurality of genes, wherein said plurality of genes comprises one or more genes selected from Table 2, a container for holding a biological sample comprising breast cancer cells isolated from a human subject for testing, and printed instructions for reacting the agents with the biological sample or a portion of the biological sample to determine if the subject is at low risk of recurrence of the breast cancer and can be spared treatment with adjuvant chemotherapy and endocrine therapy. The agents may be packaged in separate containers. In yet other embodiments, the kit may further comprise one or more control reference samples or other reagents for measuring gene expression (e.g., reagents for performing PCR, RT-PCR, microarray analysis, a Northern blot, an immunoassay, or immunohistochemistry). In some embodiments, the kit comprises agents for measuring the levels of expression of a plurality of genes listed in Table 2. In other embodiments, the kit comprises agents for measuring the levels of expression of all the genes listed in Table 2. For example, the kit may comprise a probe set, as described herein, for detecting a plurality of target nucleic acids, wherein the plurality of target nucleic acids comprises one or more gene sequences, or complements thereof, of genes selected from Table 2, or any combination thereof.

In other embodiments, the kit further comprises a system for determining a prognosis of a subject who has breast cancer and whether or not to treat the subject with adjuvant chemotherapy and endocrine therapy, wherein the system comprises: a) a probe set comprising a plurality of probes for detecting a plurality of target nucleic acids, wherein the plurality of target nucleic acids comprises one or more gene sequences, or complements thereof, of genes selected from Table 2; and b) a computer model or algorithm for analyzing an expression level or expression profile of the plurality of target nucleic acids hybridized to the plurality of probes in a biological sample from a subject who has breast cancer and determining if the subject is at low risk of cancer recurrence based on the expression level or expression profile and can be spared treatments with adjuvant chemotherapy and endocrine therapy.

These and other embodiments of the subject invention will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1C show performance and comparison of previously-published signatures in 765 subjects of SweBCG 91-RT. FIG. 1A shows a Forest plot depicting univariate (UVA) standardized hazard ratios (HRs) for each of the 15 previously-published gene signatures in all 765 subjects. Continuous risk scores were divided by the standard deviation to directly compare hazard ratios between scores with different distribution of values. When dividing by the standard deviation, the maximum possible HR is 2.5. Results are shown for all subjects and the distant metastasis endpoint.

FIG. 1B shows Pearson correlation and hierarchical clustering for the different signatures in all 765 subjects. A moderate to high correlation is seen for most signatures developed in or for breast cancer subjects with ER+ cancers. FIG. 1C shows a comparison of the previously-published signatures on their classification of individual subjects. Bar plots are colored to indicate what quartile the subject was scored per signature. Histological grade, time to metastasis, and subtype based on immunohistochemistry scores are also displayed for comparison. The results are shown for all 765 subjects. Samples are ordered by rank of the average of the fifteen signatures.

FIGS. 2A-2C show concordance of previously-published signatures in 765 subjects of SweBCG 91-RT in classifying which subjects are in the lowest quartile of risk. Barplots show the proportion of subjects classified in the lowest quartile with the title signature, that was also in the lowest quartile of each other signature. This analysis was performed for all 765 subjects.

FIG. 3 shows performance of previously-published signatures in 454 post-menopausal and systemically untreated subjects with ER+, HER2− cancers of SweBCG 91-RT. FIG. 3 shows a Forest plot depicting univariate (UVA) standardized hazard ratios (HRs) for each of the 15 previously-published gene signatures for the 454 post-menopausal and systemically untreated subjects with ER+, HER2− cancers. Continuous risk scores were divided by the standard deviation to directly compare of hazard ratios between scores with differently distributed values. Results are shown for the distant metastasis endpoint in post-menopausal and systemically untreated subjects with ER+, HER2− cancers.

FIGS. 4A-4C show performance of previously-published signatures in 454 post-menopausal and systemically untreated subjects with ER+, HER2− cancers of SweBCG 91-RT. FIGS. 4A-4C show a Kaplan-Meier plots for metastasis in the 454 post-menopausal subjects that did not receive systemic therapy with ER+, HER2− cancers in the SweBCG 91-RT cohort, for each of the 15 previously-published gene signatures.

FIGS. 5A and 5B show Kaplan-Meier survival analysis for Average Genomic Risk and MET141 in post-menopausal and systemically untreated subjects with ER+, HER2− cancers. Kaplan-Meier plots for metastasis in the 454 post-menopausal subjects that did not receive systemic therapy with ER+, HER2− cancers in the SweBCG 91-RT cohort for Average Genomic risk (FIG. 5A), and the MET141 signature (FIG. 5B).

FIGS. 6A and 6B show reactome pathway analysis. Reactome analysis pathway plots that indicate that cell cycle, DNA replication, and gene transcription pathways are overexpressed in the gene lists for previously-published signatures (FIG. 6A), and for the MET141 signature (FIG. 6B). The analysis shows that MET141 captures largely the same pathways as the previous signatures.

FIG. 7. Subject flow-chart. Flow-chart depicting SweBCG91-RT tumor samples included for gene expression analysis and in the final subgroup analysis.

FIGS. 8A-8C show Kaplan-Meier survival analysis for all subjects, metastasis endpoint. Kaplan-Meier plots showing time-to-metastasis in the full SweBCG91-RT cohort, for each of the 15 previously published gene signatures.

FIG. 9 shows a Forest plot for univariate hazard ratios, all subjects and breast cancer-specific survival endpoint. Forest plot depicting univariate (UVA) standardized hazard ratios (HRs) for each of the 15 previously published gene signatures for endpoint breast cancer-specific survival. Scores were divided by the standard deviation in order to compare between scores with different range values. Results are shown for all subjects.

FIGS. 10A-10C show Kaplan-Meier survival analysis for all subjects, BCSS endpoint. Kaplan-Meier plots showing breast cancer-specific survival in the full SweBCG91-RT cohort, for each of the 15 previously published gene signatures.

FIGS. 11A-11C show AUC over time for 15 signatures, all subjects, metastasis endpoint. Area under the curve (AUC) in a receiver operating characteristics (ROC) analysis for different timepoints. A clear decrease in performance over time is seen for most signatures indicating that they are generally better at predicting early recurrences.

FIG. 12 shows a Forest plot for univariate hazard ratios for postmenopausal and systemically untreated subjects with ER+, HER2− cancers, breast cancer-specific survival. Forest plot depicting univariate (UVA) standardized hazard ratios (HRs) for each of the 15 previously published gene signatures for endpoint breast cancer-specific survival. Scores were divided by the standard deviation in order to compare between scores with different range values. Results are shown for the 454 postmenopausal and systemically untreated subjects with ER+, HER2− cancers.

FIGS. 13A-13C show a Kaplan-Meier survival analysis, postmenopausal and systemically untreated subjects with ER+, HER2− cancers, breast cancer-specific survival. Kaplan-Meier plots showing breast cancer-specific survival in the 454 post-menopausal subjects that did not receive systemic therapy with ER+, HER2− cancers in the SweBCG91-RT cohort, for each of the 15 previously published gene signatures.

FIGS. 14A-14C show AUC over time for 15 signatures, postmenopausal and systemically untreated subjects with ER+, HER2− cancers, metastasis endpoint. Area under the curve (AUC) in a receiver operating characteristics (ROC) analysis for different timepoints. A clear decrease in performance over time is seen for most signatures meaning that they are generally better at predicting early recurrences.

FIGS. 15A-15C show a comparison of concordance in classifying the subjects as low-risk. Concordance of the signatures in classifying which subjects are in the lowest quartile of risk. Bar plots show the proportion of subjects classified in the lowest quartile with the title signature, that was also in the lowest quartile of each other signature. This analysis is performed for postmenopausal and systemically untreated subjects with ER+, HER2− cancers.

### DETAILED DESCRIPTION OF THE INVENTION

The present invention discloses systems and methods for diagnosing, predicting, and/or monitoring the status or outcome of a breast cancer in a subject using expression-based analysis of a plurality of targets. Generally, the method comprises (a) optionally providing a sample from a subject; (b) assaying the expression level for a plurality of targets in the sample; and (c) diagnosing, predicting and/or monitoring the status or outcome of a breast cancer based on the expression level of the plurality of targets.

Assaying the expression level for a plurality of targets in the sample may comprise applying the sample to a microarray. In some instances, assaying the expression level may comprise the use of an algorithm. The algorithm may be used to produce a classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

Further disclosed herein are methods for determining if the subject is at low risk of recurrence of the breast cancer. Generally, the method comprises: (a) providing a sample comprising breast cancer cells from a subject; (b) assaying the expression level for a plurality of targets in the sample; and (c) determining if the subject is at low risk of recurrence of the breast cancer based on the expression level of the plurality of targets and whether or not to treat the subject with adjuvant chemotherapy and endocrine therapy. For example, a subject identified as being at low risk of recurrence of the breast cancer may be spared treatment with adjuvant chemotherapy and endocrine therapy, whereas a subject identified as being at higher risk of recurrence of the breast cancer may be administered adjuvant chemotherapy and endocrine therapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### Targets

In some instances, assaying the expression level of a plurality of genes comprises detecting and/or quantifying a plurality of target analytes. In some embodiments, assaying the expression level of a plurality of genes comprises sequencing a plurality of target nucleic acids. In some embodiments, assaying the expression level of a plurality of biomarker genes comprises amplifying a plurality of target nucleic acids. In some embodiments, assaying the expression level of a plurality of biomarker genes comprises conducting a multiplexed reaction on a plurality of target analytes.

The methods disclosed herein often comprise assaying the expression level of a plurality of targets. The plurality of targets may comprise coding targets and/or non-coding targets of a protein-coding gene or a non-protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non-protein-coding gene structure may comprise an exon and intron. Usually, the exon region of a non-protein-coding gene primarily contains a UTR. The non-protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets comprises one or more targets selected from Table 2. In some instances, the plurality of targets comprises at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 targets selected from Table 2.

In some embodiments, the plurality of targets are selected from the group consisting of ATP binding cassette subfamily F member 1 (ABCF1), acetyl-CoA acetyltransferase 2 (ACAT2), actin like 6A (ACTL6A), apolipoprotein B mRNA editing enzyme catalytic subunit 3B (APOBEC3B), anti-silencing function 1B histone chaperone (ASF1B), ATP synthase, H+ transporting, mitochondrial F1 complex, gamma polypeptide 1 (ATP5C1), bystin like (BYSL), chromosome 1 open reading frame 106 (C1orf106), cell division cycle 25B (CDC25B), cell division cycle 45 (CDC45), chromatin licensing and DNA replication factor 1 (CDT1), CCAAT/enhancer binding protein gamma (CEBPG), centromere protein I (CENPI), chromatin assembly factor 1 subunit A (CHAF1A), chromatin assembly factor 1 subunit B (CHAFIB), checkpoint kinase 1 (CHEKI), cytokine induced apoptosis inhibitor 1 (CIAPIN1), cytoskeleton associated protein 2 (CKAP2), CTP synthase 1 (CTPS1), DNA replication helicase/nuclease 2 (DNA2), DNA methyltransferase 1 (DNMT1), DNA methyltransferase 3 beta (DNMT3B), E2F transcription factor 8 (E2F8), emopamil binding protein (sterol isomerase) (EBP), eukaryotic translation initiation factor 4E binding protein 1 (EIF4EBP1), ER membrane protein complex subunit 8 (EMC8), family with sequence similarity 96 member B (FAM96B), Fanconi anemia complementation group A (FANCA), F-box protein 5 (FBXO5), glyceraldehyde-3-phosphate dehydrogenase (GAPDH), glycyl-tRNA synthetase (GARS), geminin, DNA replication inhibitor (GMNN), G-protein signaling modulator 2 (GPSM2), GTP binding protein 4 (GTPBP4), hepatoma-derived growth factor (HDGF), hematological and neurological expressed 1 (HN1), heat shock protein family A (Hsp70) member 14 (HSPA14), heat shock protein family D (Hsp60) member 1 (HSPD1), inositol monophosphatase 2 (IMPA2), interleukin 1 receptor associated kinase 1 (IRAKI), lysyl-tRNA synthetase (KARS), kinetochore associated 1 (KNTC1), LDL receptor related protein 8 (LRP8), minichromosome maintenance complex component 5 (MCM5), minichromosome maintenance complex component 7 (MCM7), MIS18 kinetochore protein A (MIS18A), mitochondrial ribosomal protein L15 (MRPL15), mitochondrial ribosomal protein S17 (MRPS17), mitochondrial transcription termination factor 3 (MTERF3), nuclear prelamin A recognition factor (NARF), non-SMC condensin I complex subunit D2 (NCAPD2), non-SMC condensin II complex subunit D3 (NCAPD3), NADH:ubiquinone oxidoreductase subunit A9 (NDUFA9), nuclear envelope integral membrane protein 1 (NEMP1), NME/NM23 nucleoside diphosphate kinase 1 (NME1), NOP2 nucleolar protein (NOP2), nucleoporin 205 (NUP205), origin recognition complex subunit 1 (ORC1), pyruvate dehydrogenase (lipoamide) alpha 1 (PDHA1), prenyl (decaprenyl) diphosphate synthase, subunit 1 (PDSS1), phosphoglycerate kinase 1 (PGK1), polo like kinase 4 (PLK4), purine nucleoside phosphorylase (PNP), DNA polymerase alpha 2, accessory subunit (POLA2), protein phosphatase, Mg2+/Mn2+ dependent 1G (PPMIG), peroxiredoxin 4 (PRDX4), proteasome subunit beta 4 (PSMB4), proteasome subunit beta 5 (PSMB5), proteasome 26S subunit, non-ATPase 14 (PSMD14), proteasome 26S subunit, non-ATPase 2 (PSMD2), proteasome assembly chaperone 1 (PSMG1), phosphatidylserine synthase 1 (PTDSS1), RAD51 recombinase (RAD51), RAD54 homolog B (S. cerevisiae) (RAD54B), RAD54-like (S. cerevisiae) (RAD54L), RAN binding protein 1 (RANBP1), small nuclear ribonucleoprotein polypeptide A (SNRPA1), small nuclear ribonucleoprotein polypeptide G (SNRPG), SPC25, NDC80 kinetochore complex component (SPC25), SRSF protein kinase 1 (SRPK1), stress induced phosphoprotein 1 (STIP1), transforming acidic coiled-coil containing protein 3 (TACC3), transcription elongation factor B subunit 1 (TCEB1), transmembrane protein 97 (TMEM97), topoisomerase (DNA) II alpha (TOP2A), topoisomerase (DNA) II binding protein 1 (TOPBP1), triosephosphate isomerase 1 (TPI1), uracil DNA glycosylase (UNG), and tyrosine 3-monooxygenase/tryptophan 5-monooxygenase activation protein zeta (YWHAZ)

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non-protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), linear model, or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15, 20, 25, 30, 35, 40, 45, 50, or more targets selected from a Random Forest (RF) classifier. The RF classifier may be an RF2, and RF3, or an RF4 classifier. The RF classifier may be an RF22 classifier (e.g., a Random Forest classifier with 22 targets). For example, a RF classifier of the present invention may comprise two or more targets selected from Table 2.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier. The SVM classifier may be an SVM2 classifier. A SVM classifier of the present invention may comprise two or more targets selected from Table 2.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100 or more targets selected from a KNN classifier. For example, a KNN classifier of the present invention may comprise two or more targets selected from Table 2.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100 or more targets selected from a NB classifier. For example, a NB classifier of the present invention may comprise two or more targets selected from Table 2.

The plurality of targets may comprise one or more targets selected from a recursive partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100 or more targets selected from an Rpart classifier. For example, an Rpart classifier of the present invention may comprise two or more targets selected from Table 2.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15 or more targets selected from a high dimensional discriminate analysis (HDDA) classifier. For example, an Rpart classifier of the present invention may comprise two or more targets selected from Table 2.

### Probes/Primers

The present invention provides for a probe set for diagnosing, monitoring and/or predicting a status or outcome of breast cancer in a subject comprising a plurality of probes, wherein (i) the probes in the set are capable of detecting an expression level of at least one target; and (ii) the expression level determines the cancer status (e.g., risk of recurrence) of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from breast cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets. Alternatively, the probe set comprise a plurality of target sequences that hybridize to at least about 10 coding targets and/or non-coding targets. In some embodiments, the probe set comprise a plurality of target sequences that hybridize to at least about 15 coding targets and/or non-coding targets. In some embodiments, the probe set comprise a plurality of target sequences that hybridize to at least about 20 coding targets and/or non-coding targets. In some embodiments, the probe set comprise a plurality of target sequences that hybridize to at least about 30 coding targets and/or non-coding targets.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the invention comprise at least 20 consecutive bases of the nucleic acid sequence of a target or a complement thereto. The polynucleotides may comprise at least 21, 22, 23, 24, 25, 27, 30, 32, 35, 40, 45, 50, or more consecutive bases of the nucleic acids sequence of a target.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

A biological sample containing breast cancer cells is collected from a subject in need of treatment for cancer to evaluate if the subject is at low risk of cancer recurrence based on an expression level or expression profile and can be spared treatment with adjuvant chemotherapy and endocrine therapy. Diagnostic samples for use with the systems and in the methods of the present invention comprise nucleic acids suitable for providing RNAs expression information. In principle, the biological sample from which the expressed RNA is obtained and analyzed for target gene expression can be any material suspected of comprising cancerous breast tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from a breast tumor biopsy.

The samples may be archival samples, having a known and documented medical outcome, or may be samples from current subjects whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P, Paramat, Paramat Extra, Paraplast, Paraplast X-tra, Paraplast Plus, Peel Away Paraffin Embedding Wax, Polyester Wax, Carbowax Polyethylene Glycol, Polyfin, Tissue Freezing Medium TFMFM, Cryo-Gef, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 2, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 2, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from subjects showing no evidence of disease, as well as tissue and/or nucleic acids obtained from or representative of tumor samples from subjects that develop systemic cancer.

The nucleic acids may be provided in an array format, and thus an array or microarray may be included in the kit. The kit optionally may be certified by a government agency for use in prognosing the disease outcome of cancer subjects and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target genes.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tf7, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus and SenseAmp RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Genes

Any method of detecting and/or quantitating the expression of the encoded target genes can in principle be used in the invention. The expressed target genes can be directly detected and/or quantitated, or may be copied and/or amplified to allow detection of amplified copies of the expressed target genes.

Methods for detecting and/or quantifying a target gene can include Northern blotting, sequencing, array or microarray hybridization, by enzymatic cleavage of specific structures (e.g., a Clariom S assay, ThermoFisher Scientific, an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis. Methods for detecting and/or quantifying a target gene can include gene-level expression analysis of annotated genes using microarray hybridization (e.g., Clariom S assay, ThermoFisher Scientific).

In some instances, target genes may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target gene include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target genes and generate target-specific cDNA.

### TagMan® Gene Expression Analysis

TaqMan®RT-PCR can be performed using Applied Biosystems Prism (ABI) 7900 HT instruments in a 5 1.11 volume with target gene-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target, or a product derived thereof, can be used. Desirably, an array may be specific for 5, 10, 15, 20, 25, 30 or more of transcripts of a target gene. Expression of these genes may be detected alone or in combination with other transcripts. In some embodiments, an array is used which comprises a wide range of sensor probes for breast-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognosticarray is a chip.

### Data Analysis

In some embodiments, one or more pattern recognition methods can be used in analyzing the expression level of target genes. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a subject who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a subject or class of subjects.

Assaying the expression level for a plurality of target genes may comprise the use of an algorithm or classifier. Array data can be managed, classified, and analyzed using techniques known in the art. Assaying the expression level for a plurality of gene targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm or variant iterPLIER. Variance or intensity filters can be applied to pre-process data using the RMA algorithm, for example by removing target genes with a standard deviation of <10 or a mean intensity of <100 intensity units of a normalized data range, respectively.

Alternatively, assaying the expression level for a plurality of gene targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Subtyping

Molecular subtyping is a method of classifying breast cancer into one of multiple genetically-distinct categories, or subtypes. Each subtype responds differently to different kinds of treatments, and the presence of a particular subtype is predictive of, for example, chemoresistance, higher or lower risk of recurrence, or good or poor prognosis for an individual. Differential expression analysis of a plurality of the gene targets listed in Table 2 allows for the identification of subjects at low risk of cancer recurrence who may be spared treatment with adjuvant chemotherapy and endocrine therapy. In some instances, the molecular subtyping methods of the present invention are used in combination with other biomarkers, like tumor grade and hormone levels, for analyzing the breast cancer. For example, a subject with estrogen receptor positive (ER+), human epidermal growth factor receptor 2 negative (HER2−) breast cancer, who is post-menopausal, may be more likely to have a lower risk of recurrence.

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting that a subject is at low or high risk of a recurrence. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. For example, a subject determined to be at low risk of recurrence of breast cancer based on expression profiling, as described herein, may be spared adjuvant chemotherapy or endocrine therapy. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include hormonal/endocrine therapy, surgery, chemotherapy, radiation therapy, immunotherapy/biological therapy, and photodynamic therapy.

Hormonal therapy or endocrine therapy may involve administration of hormones, such as steroid hormones or hormone antagonists to modulate the levels of certain hormones in order to arrest growth or induce apoptosis of hormone-responsive cancer cells. For example, breast cancer may be treated with a selective estrogen receptor modulator (SERM) such as, but not limited to, tamoxifen, raloxifene, toremifene and fulvestrant. Alternately or additionally, inhibitors of hormone synthesis such as aromatase inhibitors, including, but not limited to, letrozole, anastrozole, exemestane, and aminoglutethimide may be used to treat breast cancer. In some cases, hormone supplementation with progestins such as, but not limited to, megestrol acetate and medroxyprogesterone acetate may be used for the treatment of hormone-responsive, advanced breast cancer. In particular, ER+ breast cancer can be treated with either an estrogen receptor antagonist (e.g. tamoxifen) or a drug that blocks the production of estrogen such as an aromatase inhibitor (e.g. anastrozole or letrozole). Hormonal therapy may also include surgical removal of endocrine organs (e.g., orchiectomy or oophorectomy).

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, Vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are Vinca alkaloids and taxanes. Vinca alkaloids may bind to specific sites on tubulin and inhibit the assembly of tubulin into microtubules (M phase of the cell cycle). The Vinca alkaloids may be derived from the Madagascar periwinkle, Catharanthus roseus (formerly known as Vinca rosea). Examples of Vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the subject or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the subject in the same manner as a normal CT scanner. Tomotherapy machines can capture CT images of the subject's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and subject positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, subjects may receive radiation therapy to the entire brain (called whole-brain radiation therapy) in addition to SRS. SRS requires the use of a head frame or other device to immobilize the subject during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a breast tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in subjects using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat subjects with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the subject. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a subject may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to subjects first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, biologic therapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit subjects undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a subject's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, prostate and breast.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in subjects with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the subject's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a subject's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outsubject procedure. PDT may also be repeated and may be used with other therapies, such as surgery, radiation, or chemotherapy.

Extracorporeal photopheresis (ECP) is a type of PDT in which a machine may be used to collect the subject's blood cells. The subject's blood cells may be treated outside the body with a photosensitizing agent, exposed to light, and then returned to the subject. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in subjects for whom the usual treatments are not appropriate, and to relieve symptoms in subjects with non-small cell lung cancer that obstructs the airways. Porfimer sodium may also be used for the treatment of precancerous lesions in subjects with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a subject's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For subjects with systemic disease, additional treatment modalities such as adjuvant chemotherapy (e.g., docetaxel, mitoxantrone and prednisone) and systemic radiation therapy (e.g., samarium or strontium) can be designated. Such subjects would likely be treated immediately with radiation therapy in order to eliminate presumed micro-metastatic disease, which cannot be detected clinically but can be revealed by the target gene expression signature. Such subjects can also be more closely monitored for signs of disease progression.

For subjects that do not have systemic disease, localized adjuvant radiation therapy or chemotherapy (e.g., localized to breast tissue) may be administered. For subjects with no evidence of disease (NED), expression profiling and/or calculation of a risk score, as described herein, may be used to determine the risk of recurrence of the breast cancer. For subjects identified as having a low risk of recurrence of breast cancer using the methods described herein, adjuvant chemotherapy and endocrine therapy would not likely be recommended by their physicians in order to avoid treatment-related side effects such as metabolic syndrome (e.g., hypertension, diabetes and/or weight gain), osteoporosis, proctitis, incontinence or impotence. Subjects with samples consistent with NED could be designated for watchful waiting, or for no treatment.

Target genes can be grouped so that information obtained about the set of target genes in the group can be used to make or assist in making a clinically relevant judgment such as a diagnosis, prognosis, or treatment choice.

A subject report is also provided comprising a representation of measured expression levels of a plurality of target genes in a biological sample from the subject, wherein the representation comprises expression levels of target genes corresponding to any one, two, three, four, five, six, eight, ten, twenty, thirty or more of the target genes, the subsets described herein, or a combination thereof. In some embodiments, the representation of the measured expression level(s) may take the form of a linear or nonlinear combination of expression levels of the target genes of interest. The subject report may be provided in a machine (e.g., a computer) readable format and/or in a hard (paper) copy. The report can also include standard measurements of expression levels of said plurality of target genes from one or more sets of subjects with known disease status and/or outcome. The report can be used to inform the subject and/or treating physician of the expression levels of the expressed target genes, the likely medical diagnosis and/or implications, and optionally may recommend a treatment modality for the subject.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from subject samples. Alternatively, the profiles can be recorded in different representational format. A graphical recordation is one such format. Clustering algorithms can assist in the visualization of such data.

### EXAMPLES

### Example 1: Comprehensive Transcriptomic Profiling Identifies Breast Cancer Subjects Who May be Spared Adjuvant Systemic Therapy

### Introduction

In this study, we aimed to better stratify subjects with early breast cancer. Factors which can impact the ability of gene expression signatures to successfully stratify subjects by risk include the lack of long-term follow-up for large well-defined subject cohorts, the lack of subject cohorts without any systemic treatment, and limited ability to compare results against those obtained using other expression signatures. Thus, this study was designed to address these problems by comprehensively profiling the transcriptome of 765 early breast cancer subjects from the SweBCG 91-RT trial, of which 703 were systemically untreated in the adjuvant setting. (Sjostrom M et al. J Clin Oncol 35:3222-3229, 2017.) Fifteen different gene expression signatures previously developed for ER+ subjects were calculated and assessed. The performance and discordance of these signatures was compared in identifying a low-risk group of subjects that could potentially be spared adjuvant chemotherapy and endocrine therapy. To alleviate the problem of discordant signatures, the signatures were combined into an Average Genomic Risk (AGR). Finally, an associated novel 141-gene signature was developed that captures the same biology with fewer genes and similar performance.

### Results

### Characteristics of the Cohort

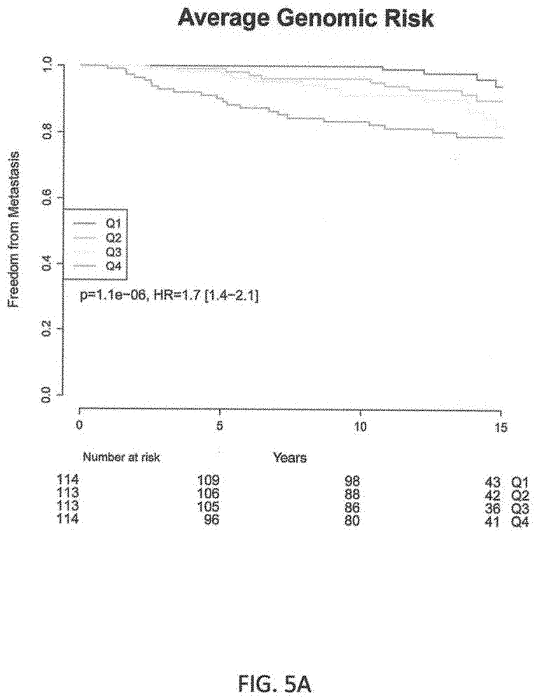

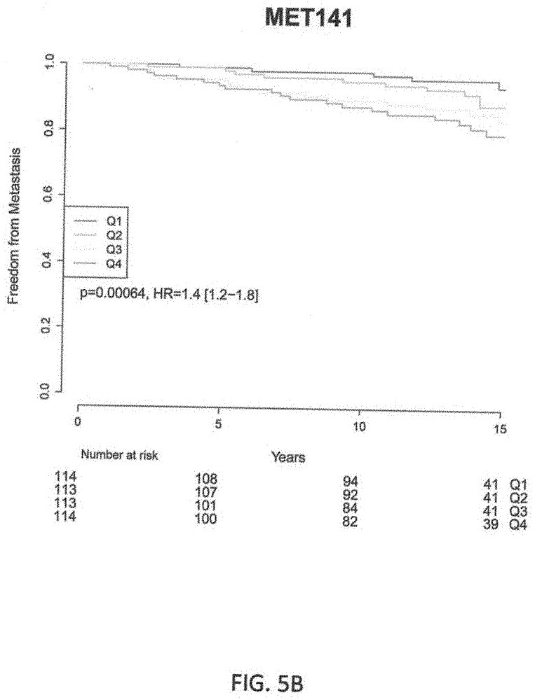

The transcriptome of 765 subjects of the SweBCG 91-RT cohort was profiled (FIG. 5). The clinical characteristics are detailed in Table 1. The cohort was enriched for ER+ and HER2− (83%) tumors and 92% of subjects did not receive adjuvant endocrine therapy or chemotherapy. In the full cohort, 84% of subjects were free of a metastasis event at 15 years.

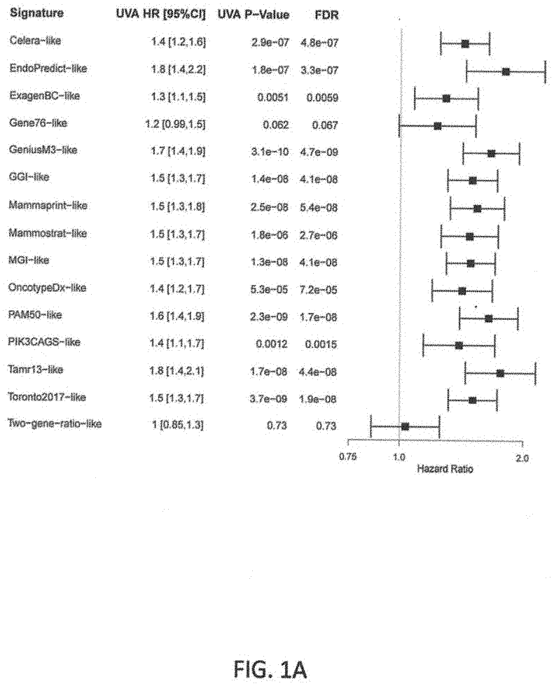

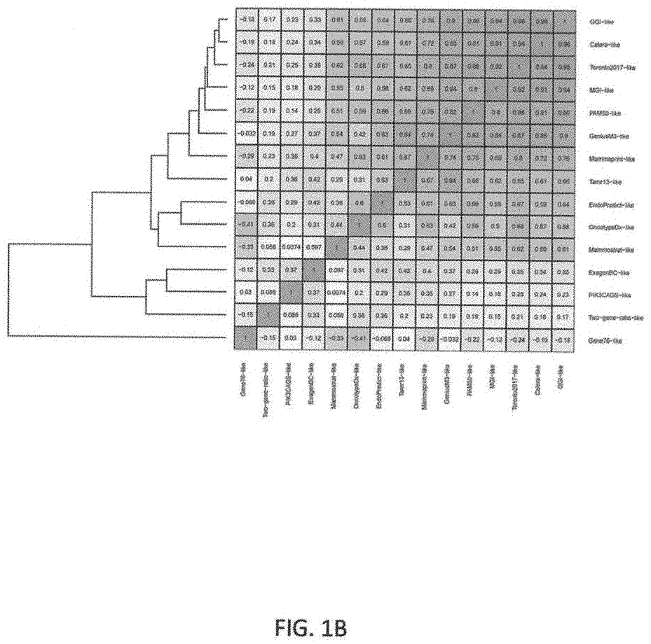

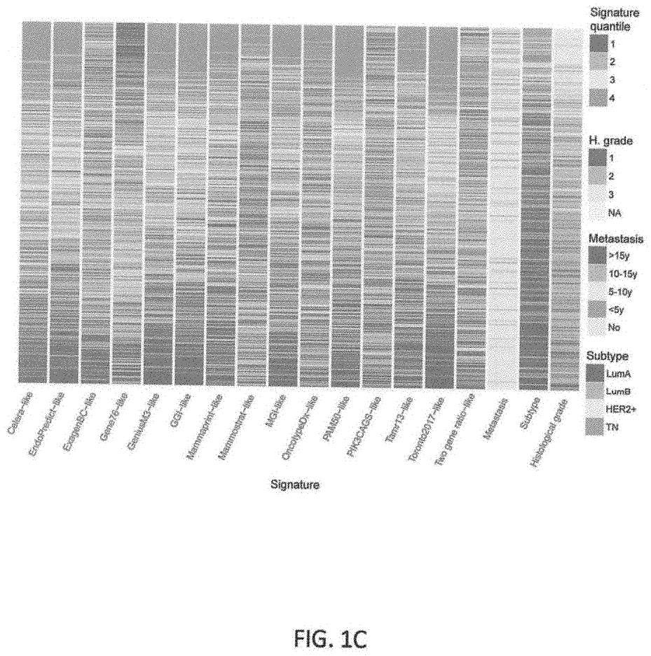

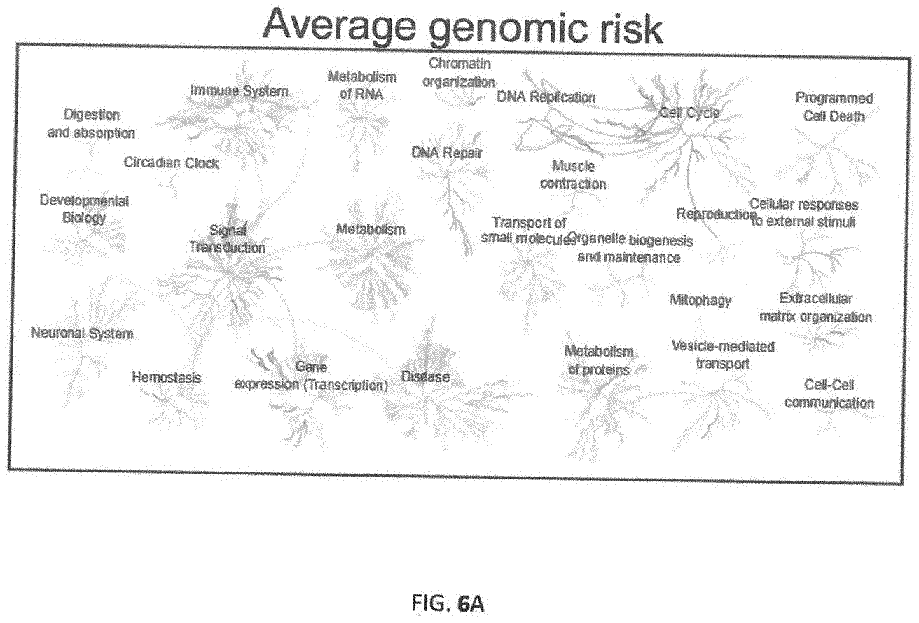

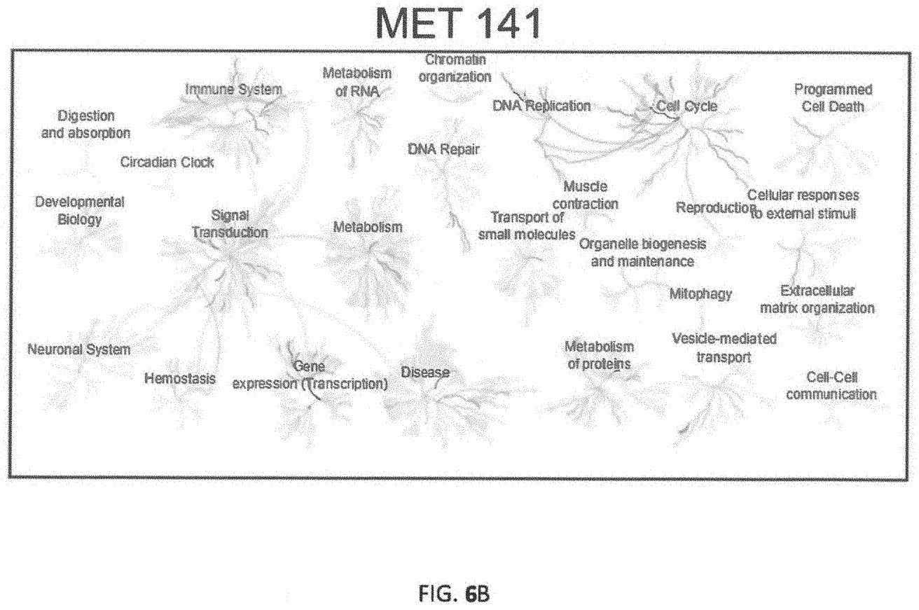

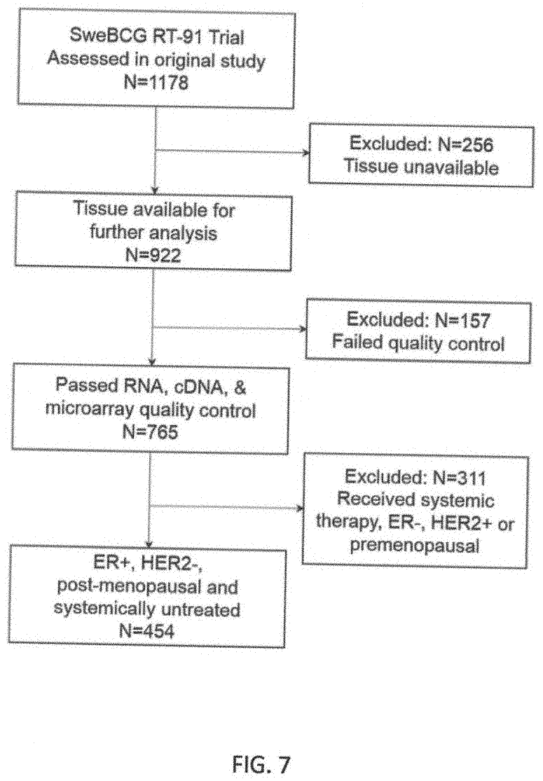

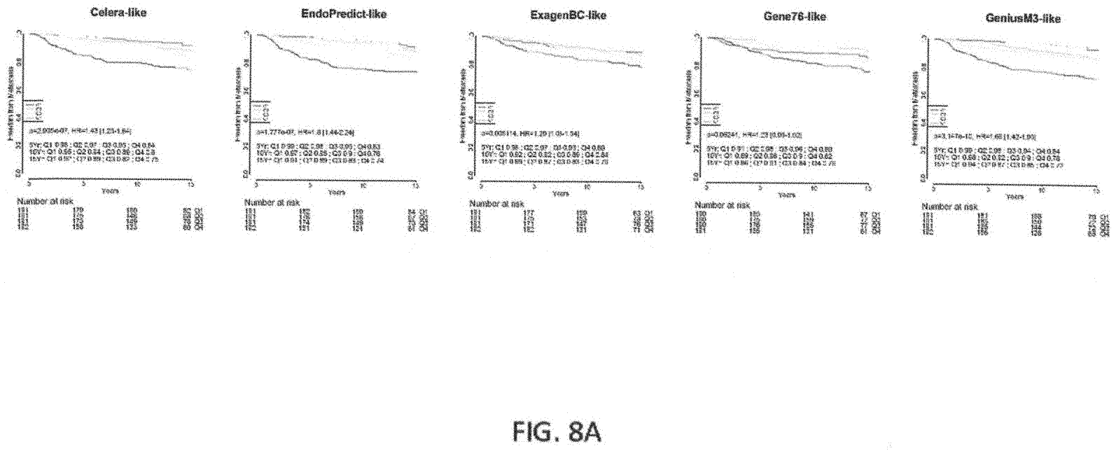

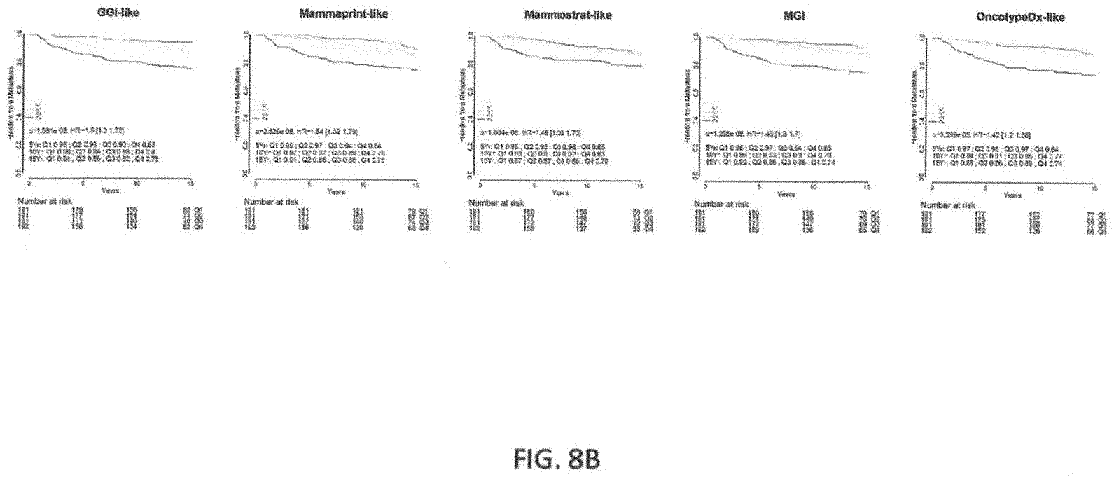

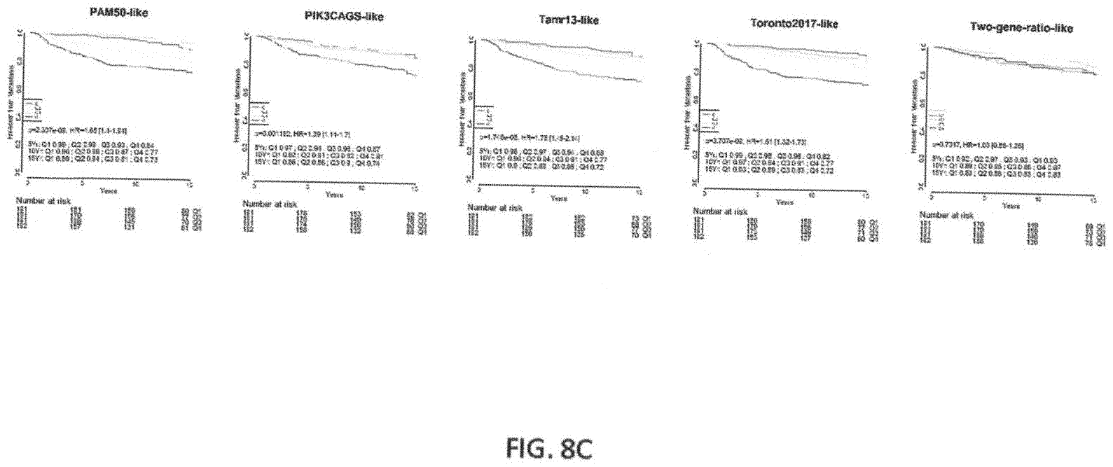

Risk scores from 15 previously-published gene expression signatures were calculated and assessed for prognostic potential for metastasis and BCSS. Thirteen of the 15 calculated scores from previously-published signatures were statistically significant (FDR<0.05) with respect to the metastasis endpoint (FIG. 1A and FIG. 6), with similar results for BCSS (FIGS. 7A-7B) and most of the continuous risk scores were highly correlated with each other (FIG. 1B). Despite high correlation of signatures, there was considerable disagreement across signatures for an individual subject (FIG. 1C). When comparing risk scores with subtype and histological grade, grade 3 and the triple-negative and HER2-subtypes had higher risk compared to grades 1-2 and the luminal subtypes (FIG. 1C). Also, subjects developing early recurrences tended to be classified as higher risk by most continuous risk scores (FIG. 1C), and signatures were better at identifying early recurrences, as shown by a higher AUC for all prognostic signatures for early recurrences (5-year) than for late recurrences (15-year) (FIG. 8).

To further evaluate the concordance of the 15 signatures for identifying low-risk subjects, we calculated the low-risk classification agreement, which quantifies the mean proportion of subjects classified in the lowest quartile of risk by one signature also classified in the lowest quartile of risk by the other signatures. Mean classification agreement ranged from 28% to 53% (FIG. 1D).

TABLE 1
Subject characteristics

| | | | ER+, HER2−, | |
|---|---|---|---|---|
| | | | post-menopausal, | |
| | All subjects | | no systemic treatment | |
| Number of subjects | 765 | | 454 | |
| Age at surgery | | | | |
| Median (range) | 59 | (31-78) | 63 | (39-78) |
| ≤39 | 19 | (3%) | 1 | (0%) |
| 40-49 | 137 | (18%) | 16 | (4%) |
| 50-59 | 234 | (31%) | 151 | (33%) |
| 60-69 | 284 | (37%) | 210 | (46%) |
| ≥70 | 91 | (12%) | 76 | (17%) |
| Menopausal status | | | | |
| Premenopausal | 152 | (20%) | 0 | (0%) |
| Post-menopausal | 592 | (80%) | 454 | (100%) |
| Missing | 21 | | 0 | |
| Histological grade | | | | |
| 1 | 105 | (14%) | 73 | (16%) |
| 2 | 457 | (61%) | 312 | (70%) |
| 3 | 191 | (25%) | 61 | (14%) |
| Missing | 12 | | 8 | |
| Tumor size (mm) | | | | |
| Median (range) | 12 | (1-40) | 11 | (1-30) |
| ≤10 | 274 | (36%) | 198 | (43%) |
| 11-20 | 415 | (55%) | 243 | (54%) |
| 21-30 | 70 | (9%) | 10 | (2%) |
| ≥31 | 1 | (0%) | 0 | (0%) |
| Missing | 5 | | 3 | |
| Estrogen receptor | | | | |
| status (>=1% by IHC) | | | | |
| Negative | 89 | (12%) | 0 | (0%) |
| Positive | 672 | (88%) | 454 | (100%) |
| Missing | 4 | | 0 | |
| Progesterone receptor | | | | |
| status (>=20% by IHC) | | | | |
| Negative | 206 | (0.269) | 0 | (0%) |
| Positive | 555 | (0.725) | 454 | (100%) |
| Missing | 4 | (0.005) | 0 | |
| HER2 status by IHC and FISH | | | | |
| Negative | 702 | (93%) | 454 | (100%) |
| Positive | 54 | (7%) | 0 | (0%) |
| Missing | 9 | | 0 | |
| Subtype by IHC | | | | |
| Luminal A | 421 | (56%) | 287 | (63%) |
| Luminal B (HER2−) | 216 | (29%) | 167 | (37%) |
| HER2+ | 54 | (7%) | 0 | (0%) |
| Triple-Negative | 65 | (9%) | 0 | (0%) |
| Missing | 9 | | 0 | |
| Adjuvant endocrine therapy | | | | |
| No | 710 | (93%) | 454 | (100%) |
| Yes | 55 | (7%) | 0 | (0%) |
| Adjuvant chemotherapy | | | | |
| No | 755 | (99%) | 454 | (100%) |
| Yes | 10 | (1%) | 0 | (0%) |
| Adjuvant radiotherapy | | | | |
| No | 403 | (53%) | 227 | (50%) |
| Yes | 362 | (47%) | 227 | (50%) |
| Distant metastasis | | | | |
| No | 658 | (86%) | 402 | (89%) |
| Yes | 107 | (14%) | 52 | (12%) |
| Died from breast cancer | | | | |
| No | 628 | (82%) | 373 | (82%) |
| Yes | 137 | (18%) | 81 | (18%) |

Performance of Calculated Scores from 15 Previously-Published Signatures in Potential Candidates for Omission of Systemic Adjuvant Treatment

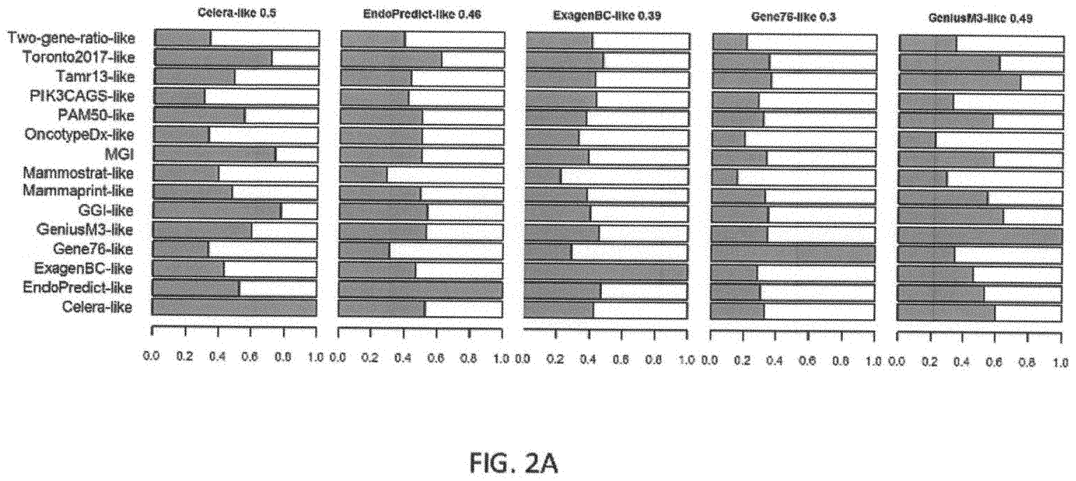

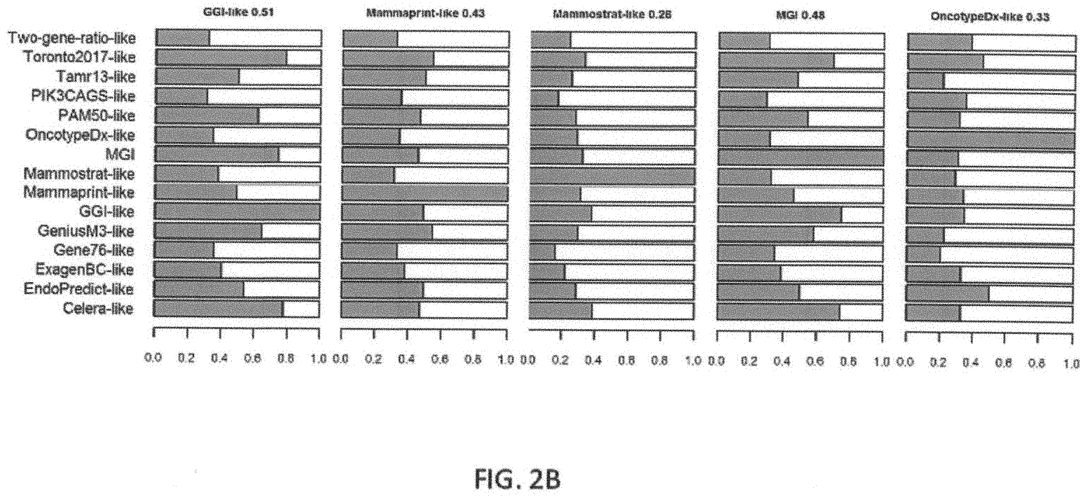

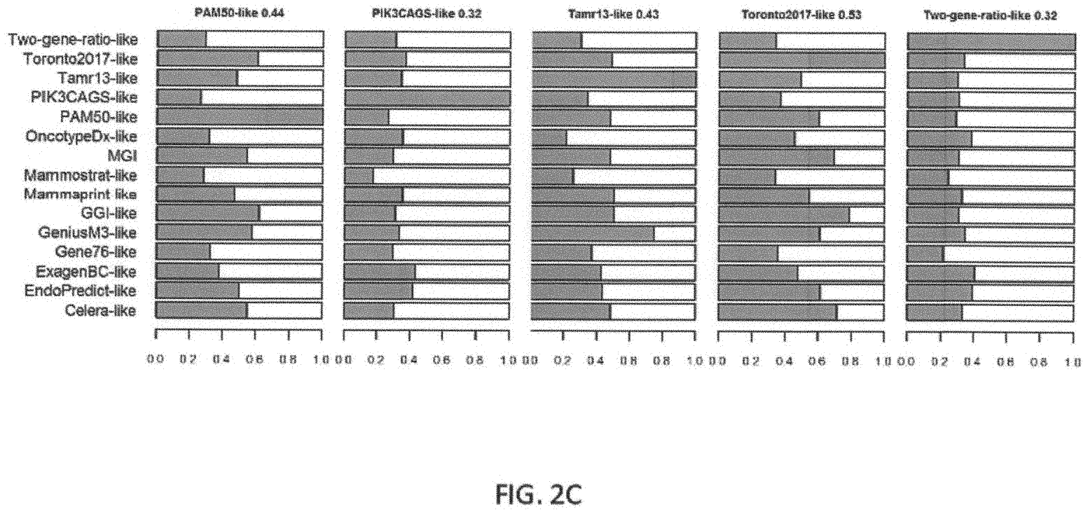

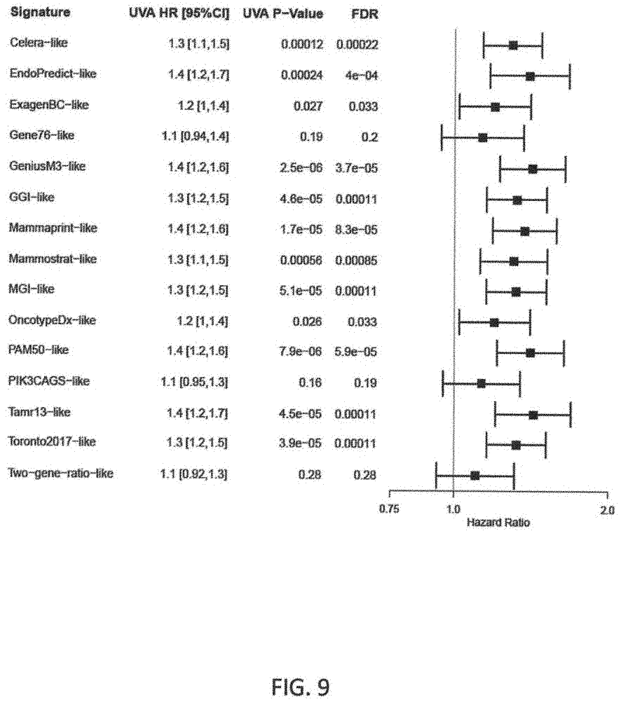

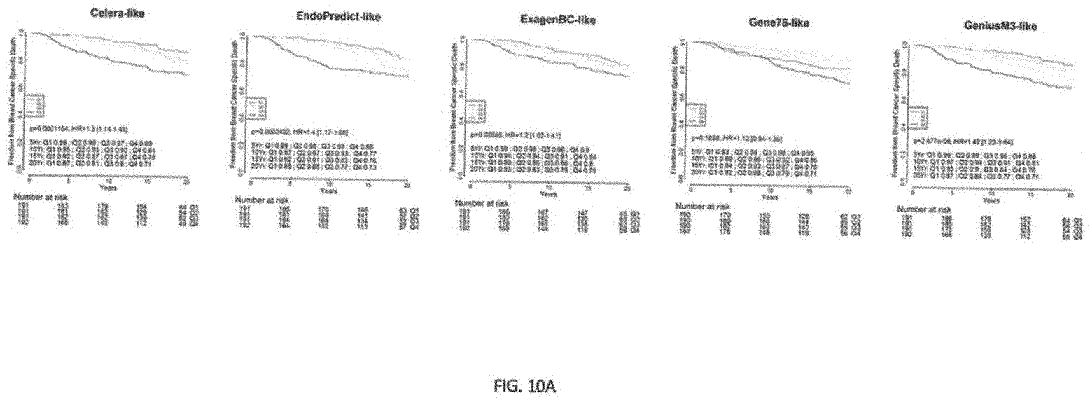

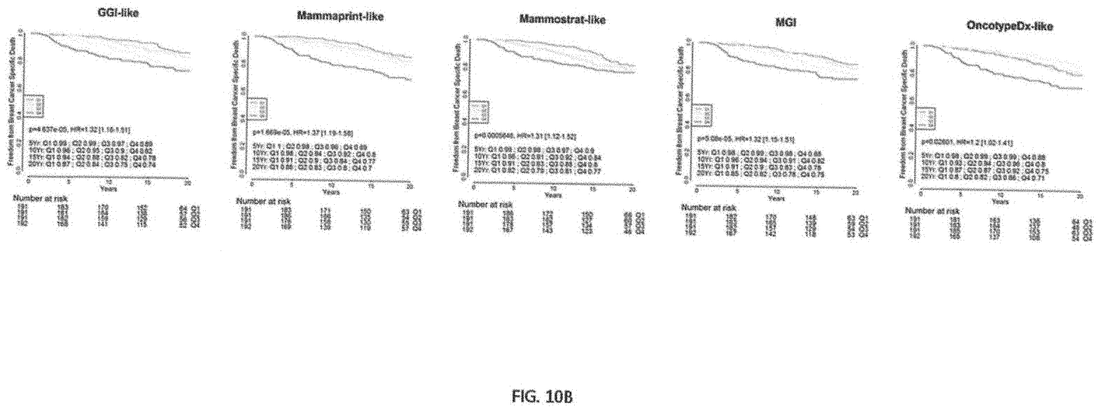

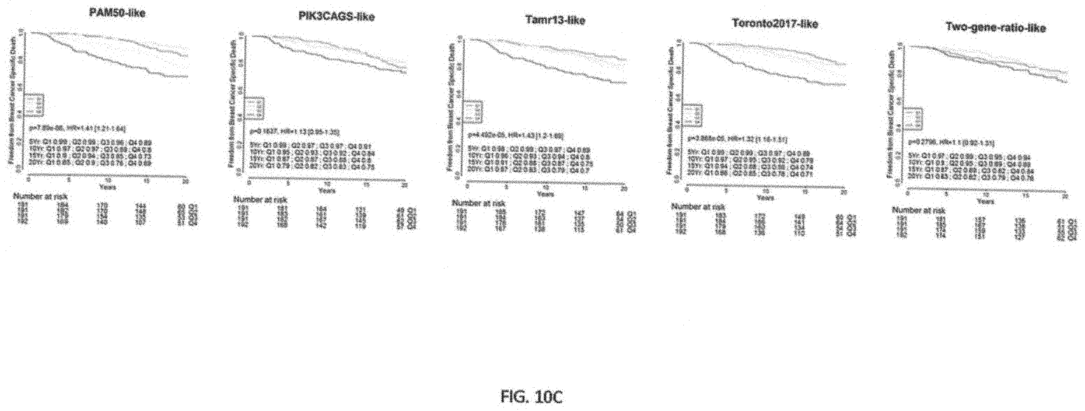

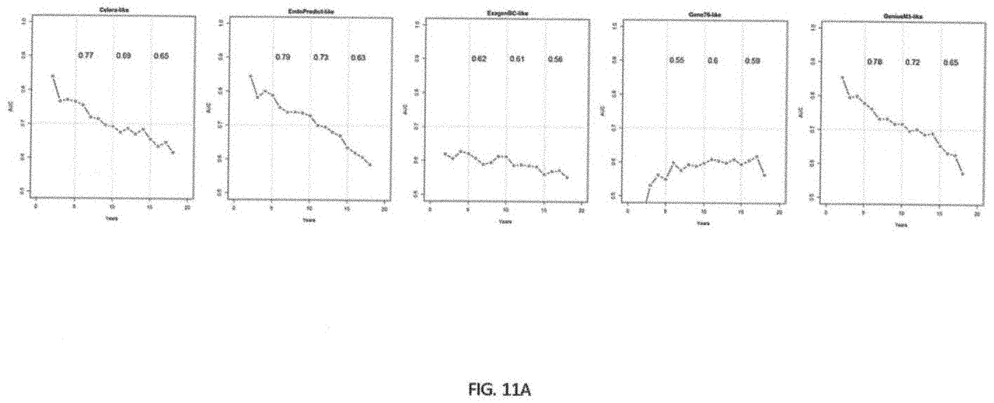

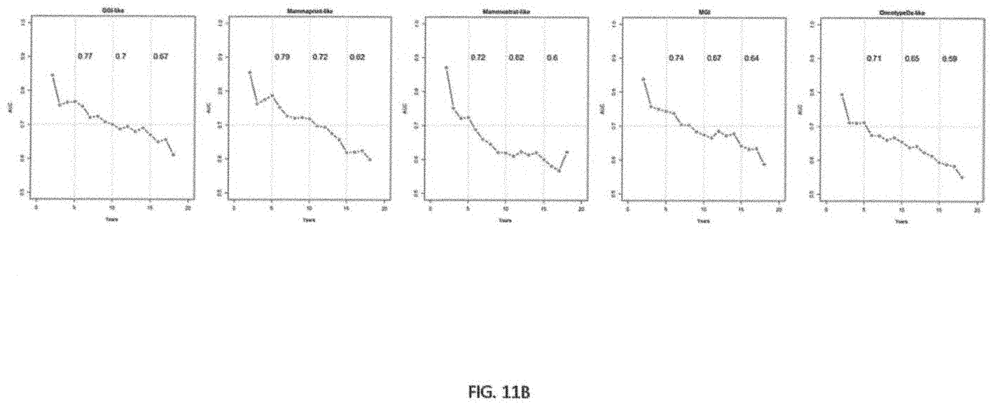

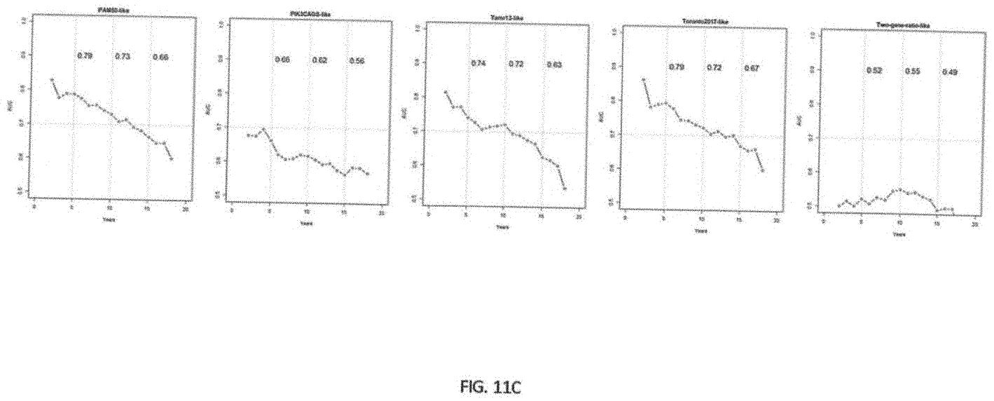

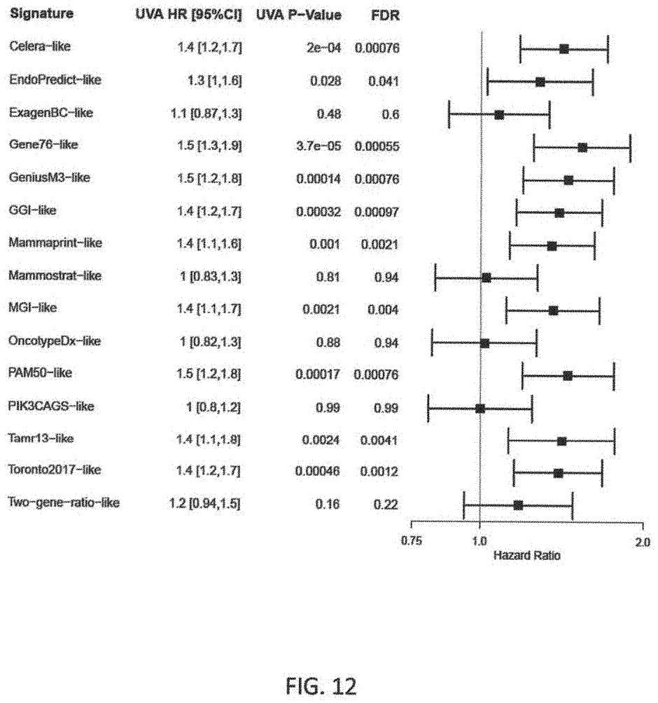

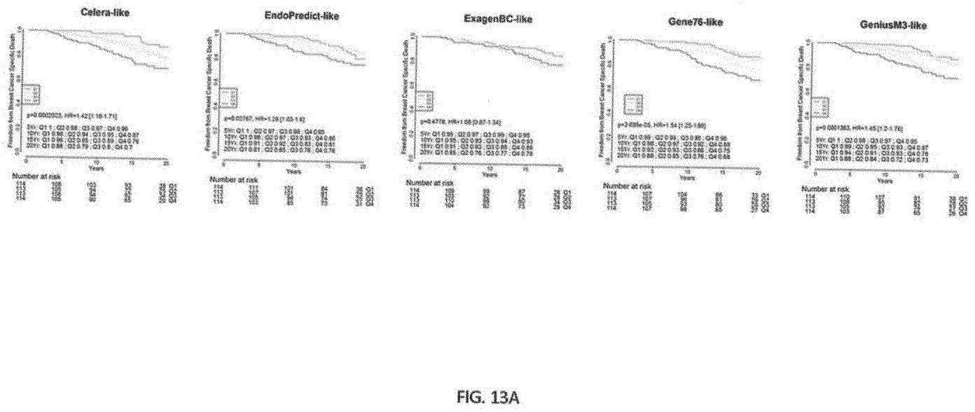

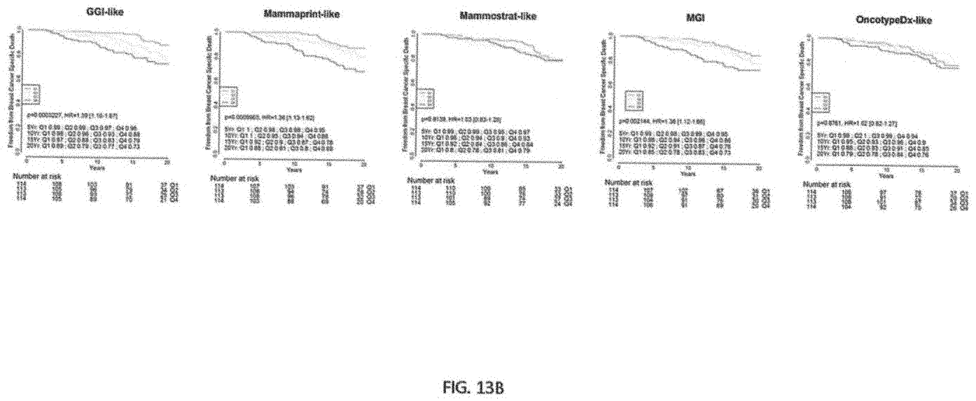

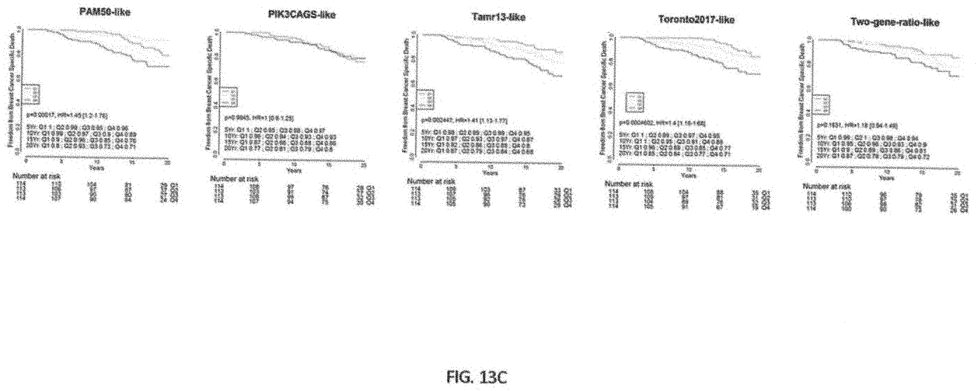

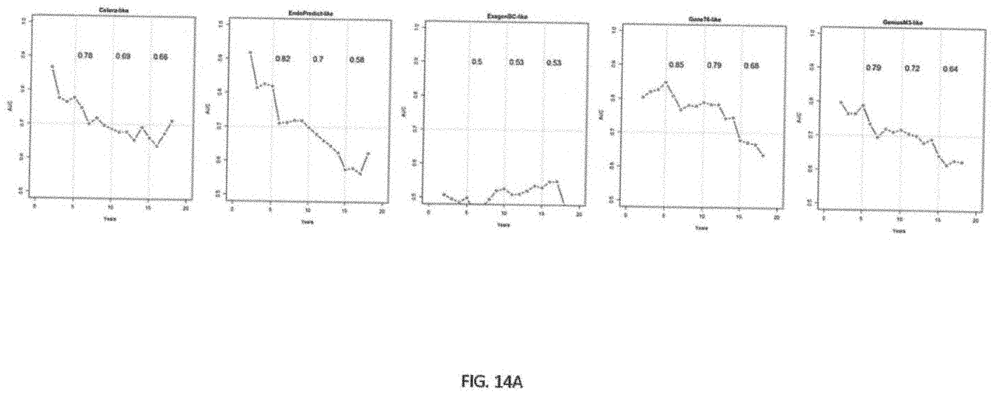

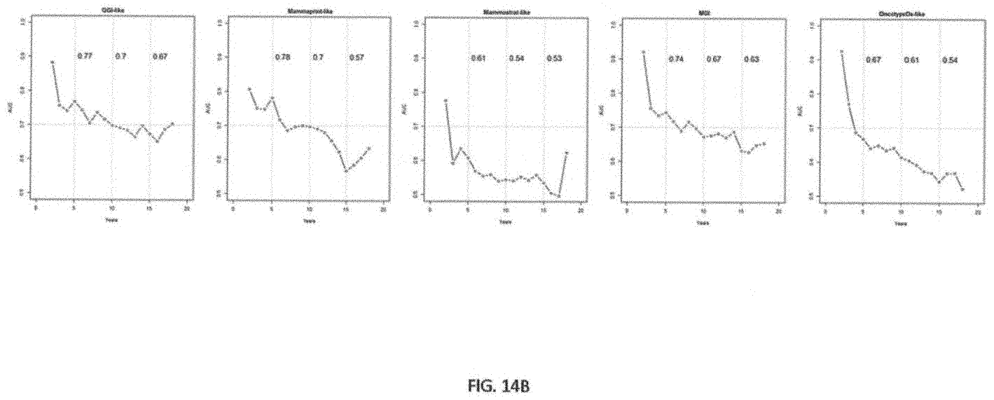

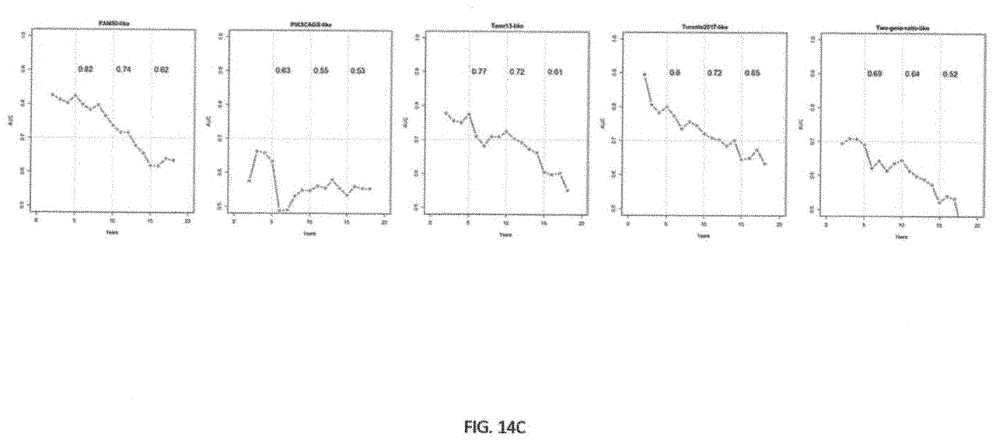

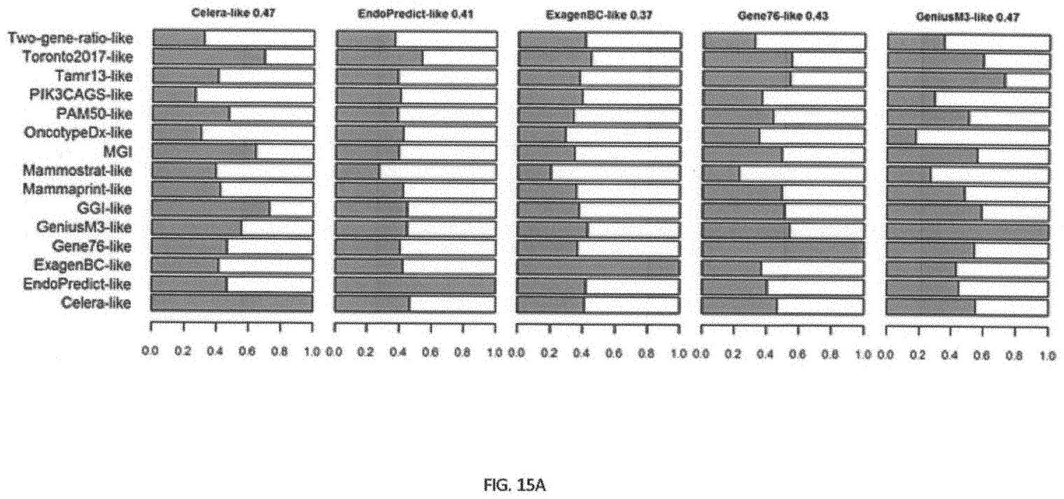

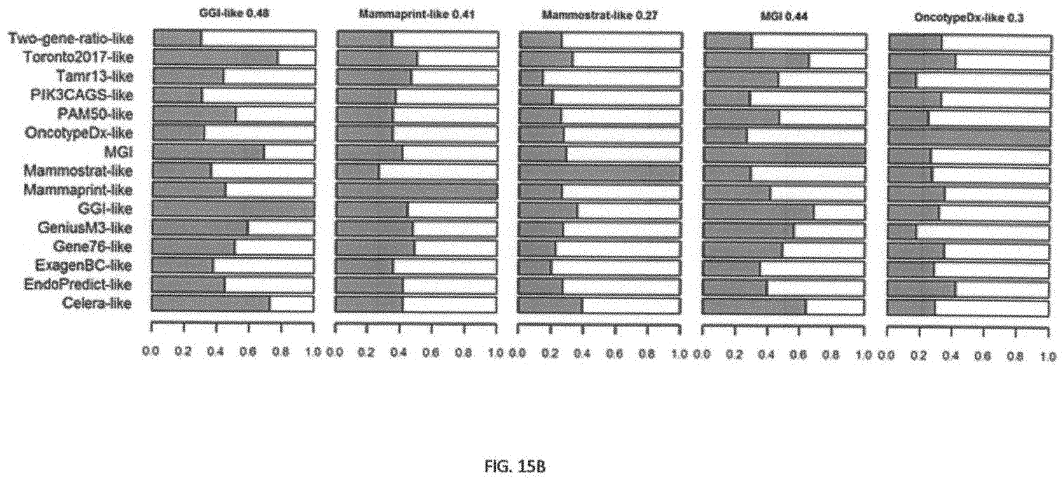

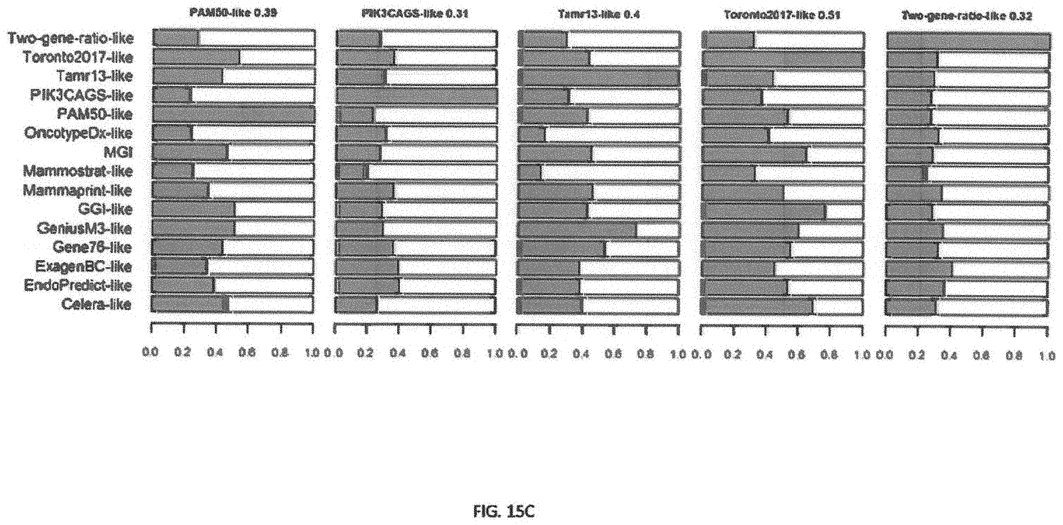

To focus on subjects who could be clinical candidates for omission of systemic adjuvant treatment, we selected subjects with ER+, HER2− tumors who were post-menopausal and did not receive any systemic adjuvant treatment (N=454, 59% of the profiled cohort). In this low-risk subgroup, 86% of subjects were free of metastasis at 15 years. Ten of the 15 signatures were significantly associated with metastasis (FDR<0.05), with scaled hazard ratios (HRs) of 1.4 to 2.1 (FIGS. 2A-2B), and the same set of signatures were also prognostic for BCSS (FIGS. 9A-9B). As risk of late recurrences is a major concern for breast cancer subjects, we analyzed the performance of the different signatures by calculating the AUC at different timepoints. For most signatures, there was a drop in prognostic ability over time (FIG. 10), with an average AUC of 0.73, 0.66, and 0.60 at 5, 10 and 15 years, respectively. The mean low-risk classification agreement between signatures varied from 27% to 51% (FIG. 11).

### Average Genomic Risk

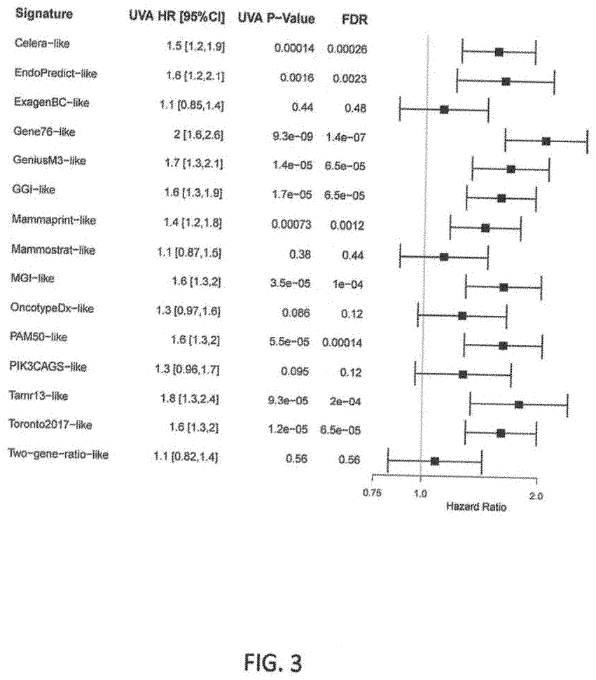

To increase the stability of the prognostication, we calculated the Average Genomic Risk (AGR) as the mean of the 15 signatures scores. The prognostic performance of AGR was in line with the most prognostic individual genomic signatures (HR=1.7 [1.4-2.1], p<0.001 for metastasis in the low-risk cohort). Furthermore, the AGR identified a very low-risk population of subjects within the ER+, HER2−, post-menopausal and systemically untreated subgroup, as subjects with the lowest quartile of AGR scores (N=114, 25% of the subgroup) had no distant metastatic event within the first 10 years, and the proportion of subjects free of metastasis at 15 years was 94% (95% CI 89-100%) (FIG. 3A).

### Signature Comparison and Related 141-Gene Signature

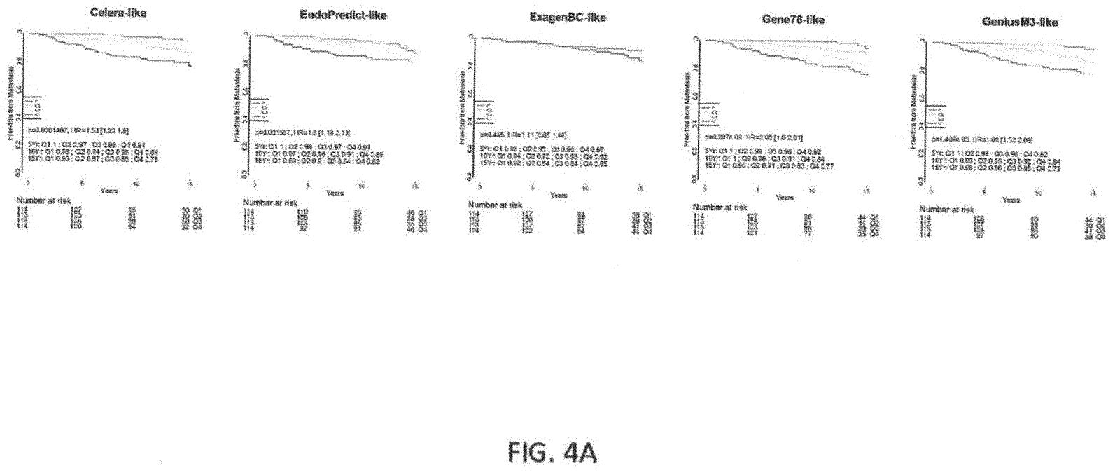

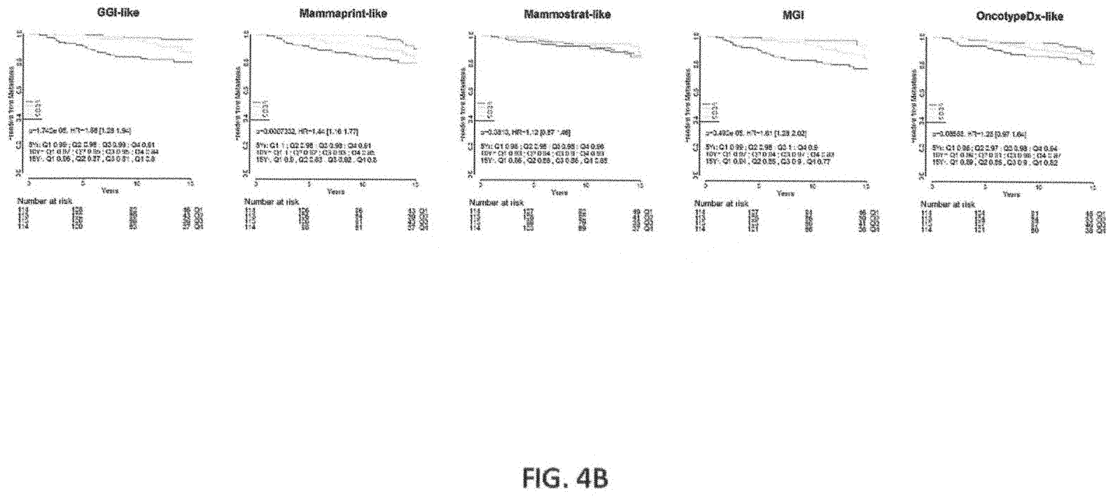

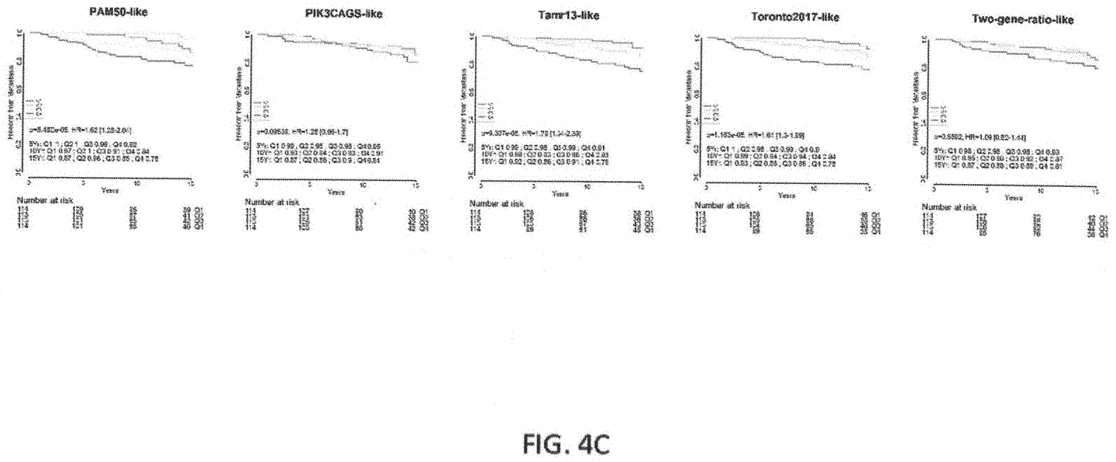

Since many of the signatures were significantly associated with time to metastasis, we aimed to identify similarities between the signatures. We performed an assessment of genes that were shared between signatures, finding that up to 100% of genes in one signature (the MGI signature, comprised of five genes) could be found in another (Table 3). When removing the Toronto 2017 signature from this analysis, as it had been derived using gene lists from many of the signatures included in this present work, and the MGI signature, which has a small total number of genes, we found that at most 69% of genes in one signature were in common with others. When performing enrichment analysis for the previously-published signatures individually, we found that cell cycle and metabolic pathways were significantly and highly enriched in these signature gene lists (FDR<0.05, Table 4). Given the similarities of gene list composition between signatures, we investigated if a signature that did not heavily share genes with these previously-published signatures could still be prognostic in this dataset. To that end, we derived a signature in five publicly available cohorts by identifying genes highly correlated with AGR but excluding overlapping genes with previous signatures. This resulted in a 141-gene signature (MET141, Table 2) with a similar performance as the AGR: 94% (95% CI 88-100%) free of metastasis at 15 years for the lowest risk quartile in the subgroup (FIG. 3B). Gene network analysis of the AGR and MET141 gene lists suggested that both were enriched in similar gene sets with a focus on cell cycle control, DNA replication, transcription and extracellular matrix organization (FIGS. 4A-4B).

### Discussion

Comprehensive transcriptomic profiling was used to evaluate the prognostic performance of 15 previously-described prognostic breast cancer signatures in SweBCG 91-RT, a unique cohort of systemically untreated subjects with long-term follow-up. These results showed that although most signatures performed well on the group level, there was considerable discordance on the individual subject level. To that end, we developed the concept of Average Genomic Risk (AGR) and an associated novel 141-gene signature (MET141). Both AGR and MET141 can identify post-menopausal and systemically untreated subjects with ER+, HER2− cancers with excellent prognosis and who may be candidates for omission of systemic therapy, including endocrine therapy. Furthermore, unlike AGR, which requires calculation and summation of risk from 15 different signatures, the MET141 signature distills similar information into a single signature.

The recent EBCTCG meta-analysis showed that late recurrences are a significant clinical problem, and that efforts to avoid endocrine therapy must rely on long-term follow-up data. (Pan H et al. N Engl J Med 377:1836-1846, 2017.) In this study, we show that the performance of calculated scores from previously-published signatures deteriorates with longer follow-up. Despite this, many of the signatures can identify a large proportion of subjects with over 90% free of metastasis at 15 years, and breast cancer-specific survival of approximately 90% at 20 years (FIGS. 6 and 3B), even without any systemic therapy.

An emerging dilemma is the considerable discordance between results of multiple gene expression tests currently in clinical use and risk prediction for individual subjects. Indeed, we have largely confirmed the results by Bartlett et al., in a different cohort, which showed only 39% of subjects classified uniformly by five tests as low-risk, while individual tests predicted a much larger proportion as low-risk. The same authors showed that three different subtyping tests disagreed for 41% of tumors. (Bartlett J M et al. J Natl Cancer Inst 108, 2016.) In our current work, we present a strategy of overcoming this by using a whole transcriptome platform and the average of all the signatures. This approach produced results consistent with the best individual signatures and could improve inter-signature variability since it relies on more data points.

Although these data suggest it may be valuable to profile tumors with all available signatures, this may not be feasible due to cost and availability of enough sample material from the tumor. To that end, we developed a novel 141-gene signature (MET141) that is based on genes correlated with AGR. The MET141 gene signature captures the same biology as the AGR and has a similar performance but would likely be more feasible in the clinical setting.

These results showed that calculated scores from previously-developed breast cancer signatures are largely prognostic in a breast cancer cohort who are post-menopausal and systemically untreated with ER+, HER2− tumors. These scores identify low-risk subjects who may be spared systemic treatment, both endocrine therapy and chemotherapy. However, the signatures are discordant on an individual subject level, and our results show that use of an average of the signatures can result in more robust subject-level results. Using this average, or an associated 141-gene signature, subjects can be identified with an excellent long-term freedom from metastasis even in the absence of any systemic treatment.

These results showed that methods and markers of the present invention are prognostic for metastasis and identify breast cancer patients who may be spared both adjuvant chemotherapy and endocrine treatment. These results further showed that the methods and markers of the present are useful for treating breast cancer.

### Subjects

A retrospective analysis of the SweBCG 91-RT trial was performed, the details of which have been previously published. (Sjostrom M et al. J Clin Oncol 35:3222-3229, 2017; Killander F et al. Eur J Cancer 67:57-65, 2016; and Malmström P et al. Eur J Cancer 39:1690-7, 2003.) Briefly, the trial randomized 1,178 node-negative, stage I and IIA subjects undergoing breast-conserving surgery to adjuvant whole breast radiation therapy or no radiotherapy. Systemic adjuvant therapy was administered according to regional guidelines at the time and was sparsely used. The original study demonstrated a benefit from radiation for locoregional events but not for breast cancer specific survival (BCSS). Subtyping was performed according to the St Gallen International Breast Cancer Conference (2013) Expert Panel as previously described. (Sjostrom M et al. J Clin Oncol 35:3222-3229, 2017.) The primary endpoint of this analysis is distant recurrence free interval (i.e., time to metastasis), defined from the time of surgery until the time of metastasis, last follow-up or death, with death as a censoring event. (Gourgou-Bourgade S et al. Ann Oncol 26:873-9, 2015.) Subjects suffering a contralateral breast cancer or another primary cancer were not censored, as recommended. (Hudis C A et al. J Clin Oncol 25:2127-32, 2007.) The median follow-up time was 14.3 years for subjects free from metastasis. For completeness, results for BCSS are shown, with a median follow-up time of 18.6 years for subjects free from event. The trial and follow-up study were approved by the Lund University ethics committee (approval numbers 2010/127 and 2015/548), and informed oral consent was obtained from all subjects.

### RNA Extraction and Gene Expression Analysis

Formalin-fixed, paraffin-embedded (FFPE) tissue samples from 922 primary tumors in the SweBCG 91-RT trial were available for further processing (FIG. 5). Cancer content was confirmed by a certified breast pathologist and a representative tumor area was marked on a hematoxylin and eosin stained slide. RNA was extracted from 1.5 mm tissue punches using the

While the preferred embodiments of the invention have been illustrated and described, it will be appreciated that various changes can be made therein without departing from the spirit and scope of the invention. RNeasy FFPE kit (Qiagen, Hilden, Germany) and cDNA was amplified using the Ovation FFPE WTA system (NuGEN, San Carlos, Calif.). Amplified cDNA was fragmented and labeled using the Encore Biotin Module (NuGEN, San Carlos, Calif.) and hybridized to GeneChip Human Exon 1.0 ST Arrays (Thermo Fisher Scientific, South San Francisco, Calif.). Sample processing was performed in a CLIA-certified clinical operations laboratory (GenomeDx Inc, San Diego, Calif.). 765 samples had sufficient RNA, and passed cDNA and microarray quality controls. Gene expression (Gene Expression Omnibus GSE119295) was normalized using Single Channel Array Normalization. (Piccolo S R et al. Genomics 100:337-44, 2012.)

### Data Analysis

### Computation of Previously-Published Breast Cancer Risk Scores

Surrogate scores of 15 previously-published gene expression signatures were calculated using published algorithms or by publicly available R packages. (Paik S et al. N Engl J Med 351:2817-26, 2004; Filipits M et al. Clin Cancer Res 17:6012-20, 2011; Sotiriou C et al. J Natl Cancer Inst 98:262-72, 2006; Parker J S et al. J Clin Oncol 27:1160-7, 2009; van de Vijver M J et al. N Engl J Med 347:1999-2009, 2002; Haibe-Kains B et al. Genome Biol 11:R18, 2010; Loi S et al. BMC Genomics 9:239, 2008; Wang Y et al. Lancet 365:671-9, 2005; Loi S et al. Proc Natl Acad Sci USA 107:10208-13, 2010; Tutt A et al. BMC Cancer 8:339, 2008; Davis L M et al. J Mol Diagn 9:327-36, 2007; Ring B Z et al. J Clin Oncol 24:3039-47, 2006; Ma X J et al. Clin Cancer Res 14:2601-8, 2008; Bayani J et al. NPJ Breast Cancer 3:3, 2017; and Ma X J et al. Cancer Cell 5:607-16, 2004.)

### Statistical Analysis

Survival analyses were performed using the R survival package (version 2.41-3). To compare hazard ratios between signatures with different ranges of values, each continuous risk score was normalized by dividing the score by its standard deviation. Estimation of time-dependent area under the curve (AUC) was calculated using the R survivalROC package (version 1.0.3). (Heagerty P J et al. Biometrics 56:337-44, 2000.) Confidence intervals for time-dependent AUC were estimated by iterating the AUC estimate over 1000 bootstrap samples to generate a sampling distribution and calculating the 2.5 and 97.5 percentiles of the resampled estimations. To compare how classification of low-risk subjects differs from signature to signature, we identified subjects within the lowest quartile of continuous risk scores of an individual signature and calculated the proportion of those subjects also classified in the lowest quartile of each other signature. We then computed the mean proportion, excluding the signature of interest (in which the proportion is 1). We refer to this as the “low-risk classification agreement”.

### Computation of Previously-Published Breast Cancer Risk Scores

Previously-published breast cancer risk scores were developed on a variety of platforms. We applied gene expression data from the GeneChip Human Exon 1.0 ST microarrays to genomic signature equations to calculate surrogate continuous risk scores. The following risk scores were calculated according to their equations as published, using the genefu package (version 2.6.0) (Gendoo D M et al. Bioinformatics 32:1097-9, 2016) in R (version 3.3.2): OncotypeDx-like, (Paik S et al. N Engl J Med 351:2817-26, 2004) Endopredict-like, (Filipits M et al. Clin Cancer Res 17:6012-20, 2011) Genomic Grade Index-like, (Sotiriou C et al. J Natl Cancer Inst 98:262-72, 2006) PAM50ROR-like, (Parker J S et al. J Clin Oncol 27:1160-7, 2009) Gene70-like, (van't Veer L J et al. Nature 415:530-6, 2002) GeniusM3-like, (Haibe-Kains B et al. Genome Biol 11:R18, 2010) TAMR-like, (Loi S et al. BMC Genomics 9:239, 2008) Gene76-like, (Wang Y et al. Lancet 365:671-9, 2005) and the PIK3CAGS-like risk score. (Loi S et al. Proc Natl Acad Sci USA 107:10208-13, 2010.) For signatures that are based on probes from specific microarrays, the genefu annotations to Entrez gene identifiers were used to map probes to the appropriate gene on our microarray platform. For the few genes not available on our microarray, the term (coefficient and gene expression value) of that gene was omitted from the signature equation. The genefu functions used are listed in Table 1.

TABLE 1
genefu functions and gene overlap for previously published signatures.

| Risk score | Genefu | |
|---|---|---|
| name | function | Genes |
| OncotypeDx-like | oncotypedx | All 16 cancer related genes are available on our |
| | | platform. |
| Endopredict-like | endoPredict | All 8 cancer related genes are available on our |
| | | platform. |
| Genomic Grade | ggi | Of 128 probes listed, 11 are not mapped to any |
| Index-like | | Entrez gene identifiers. Out of 117 listed Entrez gene |
| | | identifiers, 104 are unique and are available on our |
| | | platform. |
| PAM505ROR-like | rorS | All 50 genes are available on our platform. |
| Gene70-like | gene70 | Of the 70 probes listed, 14 are not mapped to any |
| | | Entrez gene identifier. Of the 56 listed Entrez gene |
| | | identifiers, 52 are unique and are available on our |
| | | platform. |
| GeniusM3-like | genius | All 229 genes map to our platform. |
| TAMR-like | tamrl3 | Out of the 239 unique probes, there are 169 unique |
| | | gene symbols with Entrez gene identifiers, all of |
| | | which are available on our platform. |
| PIK3CAGS-like | pik3cags | Out of 278 unique probes, there are 236 unique |
| | | Entrez gene identifiers, 234 of which map to our |
| | | platform. The two missing genes are HIST2H2AA3 |
| | | (Entrez gene ID: 8337), and KIAA0485, |
| | | uncharacterized LOC57235 (discontinued Entrez |
| | | gene ID: 57235). |
| | | In order to make the directionality of the scores in |
| | | line with the other signatures, with high scores |
| | | associated with high risk of recurrence, the final risk |
| | | score used in the study is the negative of the score |
| | | provided from genefu. |
| Gene76-like | gene76 | Out of 76 probes listed, 4 are not mapped to an |
| | | Entrez gene identifier. The remaining 72 probes map |
| | | to 67 unique genes, all of which are available on our |
| | | platform. |

### Celera-Like Risk Score

Risk scores were computed by calculating the sum of the expression of 14 genes, as previously described. (Tutt A et al. BMC Cancer 8:339, 2008.)

### ExagenBC-Like ER+ Risk Score

Risk scores were computed based on the following equation: R=0.128*CYP24−0.173*PDCD6IP+0.183*BIRC5, as previously described. (Davis L M et al. J Mol Diagn 9:327-36, 2007.)

### Mammostrat-Like Risk Score

Risk scores were computed based on the following equation: R=1.54*SLC7A5+1.12*TP53+1.06*NDRG1+0.72*HTF9C+0.5*CEACAM5, as previously described. (Ring B Z et al. J Clin Oncol 24:3039-47, 2006.)

### MGI-Like Risk Score

Risk scores were computed by normalizing the expression levels for each of the five genes in the score to have a mean of 0 and a standard deviation of 1, then combined into a single score as the first principal component, as previously described. (Ma X J et al. Clin Cancer Res 14:2601-8, 2008.)

### Toronto 2017-Like Risk Score

Risk scores were computed by calculating the linear equation involving gene expression and coefficients of 95 genes, as previously described. (Bayani J et al. NPJ Breast Cancer 3:3, 2017)

### Two Gene Ratio-Like Risk Score

Risk scores were computed by subtracting the expression of IL17RB from the expression of HOXB13, as previously described. (Ma X J et al. Cancer Cell 5:607-16, 2004.)

### Average Genomic Risk

To calculate average genomic risk, each of the fourteen signature scores was scaled from 0 to 1 within the cohort, and then the mean was computed. The scaling was necessary to prevent signatures with larger ranges of values to be over-weighted in the calculation of the average risk.

### MET141

Five publicly available breast cancer datasets were downloaded (Kreike, GE030682; Kao, GE020685; Wang, GE02034; Hatzis, GEO17705; van de Vijver, downloaded from http://microarray-pubs.stanford.edu/wound_NKI/explore.html). For each dataset, probes were converted to gene symbols, and the subset of genes in common between the five datasets were identified (10,990 genes). The average genomic risk was calculated for each subject in these five cohorts. To assess genes to include in a new signature, we removed genes in common with genes from the previously published signatures, and using the remaining 10,315 genes, correlated each gene to the average genomic risk within each cohort. Genes with a Spearman's correlation coefficient >0.4 or <−0.4 to average genomic risk in all five cohorts were retained, resulting in 141 total genes; 89 positively correlated genes and 52 negatively correlated genes (Table 2). The final MET141 score is the average expression of negatively correlated genes subtracted from the average expression of positively correlated genes.

### Pathway Analysis

To assess biological pathways overrepresented in lists of genes, we used the Panther statistical overrepresentation test (version 13.0, pantherdb.org) (Mi H et al. Nucleic Acids Res 45:D183-d189, 2017) using Fisher's Exact with FDR multiple test correction as the test type, and Panther GO-Slim Biological Process gene lists as the annotation data set. As a secondary method, we also used Reactome Analysis Tools (reactome.org) (see, Fabregat A et al. Nucleic Acids Res 46:D649-d655, 2018 and Milacic M et al. Cancers (Basel) 4:1180-211, 2012) with the “project to human” option. For both methods, lists of official gene symbols were entered. Significant enrichment of a pathway was defined as FDR<0.05.

TABLE 2
Genes included in MET141

| Genes positively correlated to higher | Genes negatively correlated to higher |
|---|---|
| genomic risk | genomic risk |
| ATP binding cassette subfamily F member | 4-aminobutyrate aminotransferase(ABAT) |
| 1(ABCF1) | |
| acetyl-CoA acetyltransferase 2(ACAT2) | actin binding LIM protein family member |
| | 3(ABLIM3) |
| actin like 6A(ACTL6A) | angiotensin II receptor type l(AGTRl) |
| apolipoprotein B mRNA editing enzyme | aldehyde dehydrogenase 6 family member |
| catalytic subunit 3B(APOBEC3B) | A1(ALDH6A1) |
| anti-silencing function 1B histone | ankyrin repeat family A member 2(ANKRA2) |
| chaperone(ASF1B) | |
| ATP synthase, H+ transporting, | BCL2 interacting protein 3 like(BNIP3L) |
| mitochondrial F1 complex, gamma | |
| polypeptide 1(ATP5C1) | |
| bystin like(BYSL) | BTG anti-proliferation factor 2(BTG2) |
| chromosome 1 open reading frame | cyclin G2(CCNG2) |
| 106(Clorf106) | |
| cell division cycle 25B(CDC25B) | cold inducible RNA binding protein(CIRBP) |
| cell division cycle 45(CDC45) | cytochrome c oxidase subunit 7A1(COX7A1) |
| chromatin licensing and DNA replication | cystatin C(CST3) |
| factor 1(CDT1) | |
| CCAAT/enhancer binding protein | C-X-C motif chemokine ligand 12(CXCL12) |
| gamma(CEBPG) | |
| centromere protein I(CENPI) | decorin(DCN) |
| chromatin assembly factor 1 subunit | enoyl-CoA hydratase domain containing |
| A(CHAF1A) | 2(ECHDC2) |
| chromatin assembly factor 1 subunit | ELOVL fatty acid elongase 5(ELOVL5) |
| B(CHAF1B) | |
| checkpoint kinase 1(CHEK1) | family with sequence similarity 13 member |
| | B(FAM13B) |
| cytokine induced apoptosis inhibitor | family with sequence similarity 198 member |
| 1(CIAPIN1) | B(FAM198B) |
| cytoskeleton associated protein 2(CKAP2) | F-box and leucine rich repeat protein 5(FBXL5) |
| CTP synthase 1(CTPS1) | flavin containing monooxygenase 5(FMO5) |
| DNA replication helicase/nuclease 2(DNA2) | gap junction protein alpha 1(GJA1) |
| DNA methyltransferase 1(DNMT1) | HtrA serine peptidase 1 (HTRA1) |
| DNA methyltransferase 3 beta(DNMT3B) | immunoglobulin (CD79A) binding protein |
| | 1(IGBP1) |
| E2F transcription factor 8(E2F8) | insulin like growth factor binding protein |
| | 4(IGFBP4) |
| emopamil binding protein (sterol | insulin like growth factor binding protein 6 |
| isomerase)(EBP) | (IGFBP6) |
| eukaiyotic translation initiation factor 4E | interleukin 20 receptor subunit alpha(IL20RA) |
| binding protein 1(EIF4EBP1) | |
| ER membrane protein complex subunit | insulin receptor substrate 1(IRS1) |
| 8(EMC8) | |
| family with sequence similarity 96 member | integral membrane protein 2B(ITM2B) |
| B(FAM96B) | |
| Fanconi anemia complementation group | inositol 1,4,5-trisphosphate receptor type |
| A(FANCA) | 1(ITPR1) |
| F-box protein 5(FBXO5) | leucine zipper transcription factor like |
| | 1(LZTFL1) |
| glyceraldehyde-3 -phosphate | mannosidase alpha class 2B member |
| dehydrogenase(GAPDH) | 2(MAN2B2) |
| glycyl-tRNA synthetase(GARS) | Meis homeobox 3 pseudogene 1(MEIS3P1) |
| geminin, DNA replication inhibitor(GMNN) | microtubule associated tumor suppressor |
| | 1(MTUS1) |
| G-protein signaling modulator 2(GPSM2) | myosin VC(MYO5C) |
| GTP binding protein 4(GTPBP4) | myoferlin(MYOF) |
| hepatoma-derived growth factor(HDGF) | neurobeachin(NBEA) |
| hematological and neurological expressed | nischarin(NISCH) |
| 1(HN1) | |
| heat shock protein family A (Hsp70) | NME/NM23 family member 5(NME5) |
| member 14(HSPA14) | |
| heat shock protein family D (Hsp60) | prolactin induced protein(PIP) |
| member 1(HSPD1) | |
| inositol monophosphatase 2(IMPA2) | prostaglandin E receptor 3(PTGER3) |
| interleukin 1 receptor associated kinase | quinoid dihydropteridine reductase(QDPR) |
| 1(IRAK1) | |
| lysyl-tRNA synthetase(KARS) | rabaptin, RAB GTPase binding effector protein |
| | 1(RABEP1) |
| kinetochore associated 1(KNTC1) | retinoic acid induced 2(RAI2) |
| LDL receptor related protein 8(LRP8) | ribonuclease A family member 4(RNASE4) |
| minichromosome maintenance complex | store-operated calcium entry associated |
| component 5(MCM5) | regulatory factor(SARAF) |
| minichromosome maintenance complex | SH3 domain binding glutamate rich protein |
| component 7(MCM7) | like(SH3BGRL) |
| MIS18 kinetochore protein A(MIS18A) | sushi domain containing 6(SUSD6) |
| mitochondrial ribosomal protein | syntabulin(SYBU) |
| L15(MRPL15) | |
| mitochondrial ribosomal protein | TBC1 domain family member 9(TBC1D9) |
| S17(MRPS17) | |
| mitochondrial transcription termination | transcription elongation factor A like |
| factor 3(MTERF3) | 1(TCEAL1) |
| nuclear prelamin A recognition | transmembrane BAX inhibitor motif containing |
| factor(NARF) | 4(TMBIM4) |
| non-SMC condensin I complex subunit | ubiquitin like 3(UBL3) |
| D2(NCAPD2) | |
| non-SMC condensin II complex subunit | zinc finger and BTB domain containing |
| D3(NCAPD3) | 16(ZBTB16) |
| NADH:ubiquinone oxidoreductase subunit | |
| A9(NDUFA9) | |
| nuclear envelope integral membrane protein | |
| 1(NEMP1) | |
| NME/NM23 nucleoside diphosphate kinase | |
| 1(NME1) | |
| NOP2 nucleolar protein(NOP2) | |
| nucleoporin 205(NUP205) | |
| origin recognition complex subunit 1(ORC1) | |
| pyruvate dehydrogenase (lipoamide) alpha | |
| 1(PDHA1) | |
| prenyl (decaprenyl) diphosphate synthase, | |
| subunit 1(PDSS1) | |
| phosphoglycerate kinase 1(PGK1) | |
| polo like kinase 4(PLK4) | |
| purine nucleoside phosphorylase(PNP) | |
| DNA polymerase alpha 2, accessory | |
| subunit(POLA2) | |
| protein phosphatase, Mg2+/Mn2+ dependent | |
| 1G(PPM1G) | |
| peroxiredoxin 4(PRDX4) | |
| proteasome subunit beta 4(PSMB4) | |
| proteasome subunit beta 5 (PSMB5) | |
| proteasome 26S subunit, non-ATPase | |
| 14(PSMD14) | |
| proteasome 26S subunit, non-ATPase | |
| 2(PSMD2) | |
| proteasome assembly chaperone 1(PSMG1) | |
| phosphatidylserine synthase 1(PTDSS1) | |
| RAD51 recombinase(RAD51) | |
| RAD54 homolog B (S. | |
| cerevisiae)(RAD54B) | |
| RAD54-like (S. cerevisiae)(RAD54L) | |
| RAN binding protein 1(RANBP1) | |
| small nuclear ribonucleoprotein polypeptide | |
| A′(SNRPA1) | |
| small nuclear ribonucleoprotein polypeptide | |
| G(SNRPG) | |
| SPC25, NDC80 kinetochore complex | |
| component(SPC25) | |
| SRSF protein kinase 1(SRPK1) | |
| stress induced phosphoprotein 1(STIP1) | |
| transforming acidic coiled-coil containing | |
| protein 3(TACC3) | |
| transcription elongation factor B subunit | |
| 1(TCEB1) | |
| transmembrane protein 97(TMEM97) | |
| topoisomerase (DNA) II alpha(TOP2A) | |
| topoisomerase (DNA) II binding protein | |
| 1 (TOPBP1) | |
| triosephosphate isomerase 1(TPI1) | |
| uracil DNA glycosylase(UNG) | |
| tyrosine 3-monooxygenase/tiyptophan | |
| 5-monooxygenase activation protein | |
| zeta(YWHAZ) | |

TABLE 3
Genes in common between previously published signatures

| | Celera-like | | Endopredict-like | | ExagenBC-like | | Gene70-like | | Gene76-like | |
|---|---|---|---|---|---|---|---|---|---|---|
| Celera-like | 14/14 | (100%) | 0/14 | (0%) | 0/14 | (0%) | 4/14 | (29%) | 0/14 | (0%) |
| Endopredict-like | 0/8 | (0%) | 8/8 | (100%) | 1/8 | (12%) | 0/8 | (0%) | 0/8 | (0%) |
| ExagenBC-like | 0/3 | (0%) | 1/3 | (33%) | 3/3 | (100%) | 0/3 | (0%) | 0/3 | (0%) |
| Gene70-like | 4/52 | (7.7%) | 0/52 | (0%) | 0/52 | (0%) | 52/52 | (100%) | 1/52 | (1.9%) |
| Gene76-like | 0/67 | (0%) | 0/67 | (0%) | 0/67 | (0%) | 1/67 | (1.5%) | 67/67 | (100%) |
| GeniusM3-like | 5/229 | (2.2%) | 2/229 | (0.87%) | 1/229 | (0.44%) | 4/229 | (1.7%) | 3/229 | (1.3%) |
| GGI-like | 7/104 | (6.7%) | 2/104 | (1.9%) | 1/104 | (0.96%) | 8/104 | (7.7%) | 6/104 | (5.8%) |
| Mammostrat-like | 0/5 | (0%) | 0/5 | (0%) | 0/5 | (0%) | 0/5 | (0%) | 0/5 | (0%) |
| MGI-like | 2/5 | (40%) | 0/5 | (0%) | 0/5 | (0%) | 1/5 | (20%) | 0/5 | (0%) |
| OncotypeDx-like | 2/16 | (12%) | 1/16 | (6.2%) | 1/16 | (6.2%) | 1/16 | (6.2%) | 0/16 | (0%) |
| PAM50-like | 4/50 | (8%) | 2/50 | (4%) | 1/50 | (2%) | 1/50 | (2%) | 0/50 | (0%) |
| PIK3CAGS-like | 0/236 | (0%) | 0/236 | (0%) | 0/236 | (0%) | 0/236 | (0%) | 6/236 | (2.5%) |
| TAMR13-like | 7/169 | (4.1%) | 2/169 | (1.2%) | 1/169 | (0.59%) | 8/169 | (4.7%) | 6/169 | (3.6%) |
| Toronto 2017-like | 9/95 | (9.5%) | 2/95 | (2.1%) | 1/95 | (1.1%) | 23/95 | (24%) | 3/95 | (3.2%) |
| Two Gene Ratio-lik | 0/2 | (0%) | 0/2 | (%) | 0/2 | (0%) | 0/2 | (0%) | 0/2 | (0%) |

| | GeniusM3-like | | GGI-like | | Mammostrat-like | |
|---|---|---|---|---|---|---|
| Celera-like | 5/14 | (36%) | 7/14 | (50%) | 0/14 | (0%) |
| Endopredict-like | 2/8 | (25%) | 2/8 | (25%) | 0/8 | (0%) |
| ExagenBC-like | 1/3 | (33%) | 1/3 | (33%) | 0/3 | (0%) |
| Gene70-like | 4/52 | (7.7%) | 8/52 | (15%) | 0/52 | (0%) |
| Gene76-like | 3/67 | (4.5%) | 6/67 | (9%) | 0/67 | (0%) |
| GeniusM3-like | 229/229 | (100%) | 34/229 | (15%) | 0/229 | (0%) |
| GGI-like | 34/104 | (33%) | 104/104 | (100%) | 1/104 | (0.96%) |
| Mammostrat-like | 0/5 | (0%) | 1/5 | (20%) | 5/5 | (100%) |
| MGI-like | 3/5 | (60%) | 4/5 | (80%) | 0/5 | (0%) |
| OncotypeDx-like | 3/16 | (19%) | 5/16 | (31%) | 0/16 | (0%) |
| PAM50-like | 9/50 | (18%) | 13/50 | (26%) | 0/50 | (0%) |
| PIK3CAGS-like | 8/236 | (3.4%) | 0/236 | (0%) | 1/236 | (0.42%) |
| TAMR13-like | 34/169 | (20%) | 41/169 | (24%) | 0/169 | (0%) |
| Toronto 2017-like | 21/95 | (22%) | 32/95 | (34%) | 3/95 | (3.2%) |
| Two Gene Ratio-lik | 0/2 | (0%) | 0/2 | (0%) | 0/2 | (0%) |

| | MGI-like | | OncotypeDx-like | | PAM50-like | | PIK3CAGS-like | |
|---|---|---|---|---|---|---|---|---|
| Celera-like | 2/14 | (14%) | 2/14 | (14%) | 4/14 | (29%) | 0/14 | (0%) |
| Endopredict-like | 0/8 | (0%) | 1/8 | (12%) | 2/8 | (25%) | 0/8 | (0%) |
| ExagenBC-like | 0/3 | (0%) | 1/3 | (33%) | 1/3 | (33%) | 0/3 | (0%) |
| Gene70-like | 1/52 | (1.9%) | 1/52 | (1.9%) | 1/52 | (1.9%) | 0/52 | (0%) |
| Gene76-like | 0/67 | (0%) | 0/67 | (0%) | 0/67 | (0%) | 6/67 | (9%) |
| GeniusM3-like | 3/229 | (1.3%) | 3/229 | (1.3%) | 9/229 | (3.9%) | 8/229 | (3.5%) |
| GGI-like | 4/104 | (3.8%) | 5/104 | (4.8%) | 13/104 | (12%) | 0/104 | (0%) |
| Mammostrat-like | 0/5 | (0%) | 0/5 | (0%) | 0/5 | (0%) | 1/5 | (20%) |
| MGI-like | 5/5 | (100%) | 0/5 | (0%) | 1/52 | (20%) | 0/5 | (0%) |
| OncotypeDx-like | 0/16 | (0%) | 16/16 | (100%) | 11/16 | (69%) | 3/16 | (19%) |
| PAM50-like | 1/50 | (2%) | 11/50 | (22%) | 50/50 | (100%) | 5/50 | (10%) |
| PIK3CAGS-like | 0/236 | (0%) | 3/236 | (1.3%) | 5/236 | (2.1%) | 236/236 | (100%) |
| TAMR13-like | 5/169 | (3%) | 4/169 | (2.4%) | 15/169 | (8.9%) | 3/169 | (1.8%) |
| Toronto 2017-like | 5/95 | (5.3%) | 10/95 | (11%) | 36/95 | (38%) | 2/95 | (2.1%) |
| Two Gene Ratio-lik | 0/236 | (0%) | 0/2 | (0%) | 0/2 | (0%) | 0/2 | (0%) |

| | TAMR13-like | | Toronto 2017-like | | Two Gene Ratio-like | |
|---|---|---|---|---|---|---|
| celera-like | 7/14 | (50%) | 9/14 | (64%) | 0/14 | (0%) |
| Endopredict-like | 2/8 | (25%) | 2/8 | (25%) | 0/8 | (0%) |
| ExagenBC-like | 1/3 | (33%) | 1/3 | (33%) | 0/3 | (0%) |
| Gene70-like | 8/52 | (15%) | 23/52 | (44%) | 0/52 | (0%) |
| Gene76-like | 6/67 | (9%) | 3/67 | (4.5%) | 0/67 | (0%) |
| GeniusM3-like | 34/229 | (15%) | 21/229 | (9.2%) | 0/229 | (0%) |
| GGI-like | 41/104 | (39%) | 32/104 | (31%) | 0/104 | (0%) |
| Mammostrat-like | 0/5 | (0%) | 3/5 | (60%) | 0/50 | (0%) |
| MGI-like | 5/5 | (100%) | 5/5 | (100%) | 0/50 | (0%) |
| OncotypeDx-like | 4/16 | (25%) | 10/16 | (62%) | 0/169 | (0%) |
| PAM50-like | 15/50 | (30%) | 36/50 | (72%) | 0/50 | (0%) |
| PIK3CAGS-like | 3/236 | (1.3%) | 2/236 | (0.85%) | 0/236 | (0%) |
| TAMR13-like | 169/169 | (100%) | 31/169 | (18%) | 0/169 | (0%) |
| Toronto 2017-like | 31/95 | (33%) | 95/95 | (100%) | 0/95 | (0%) |
| Two Gene Ratio-lik | 0/2 | (0%) | 0/2 | (0%) | 2/2 | (100%) |

TABLE 4
Overexpression analysis for previously published genomic signatures

| | Fold Enrichment | Raw P-value | FDR |
|---|---|---|---|
| Celera | | | |
| regulation of cell cycle (GO:0051726) | 22.63 | 2.99E−04 | 3.65E−02 |
| DNA metabolic process (GO:0006259) | 14.35 | 1.41E−04 | 3.45E−02 |
| GENIUSM3 | | | |
| DNA recombination (GO:0006310) | 7.23 | 2.87E−03 | 4.66E−02 |
| DNA replication (GO:0006260) | 7.18 | 2.47E−07 | 3.01E−05 |
| cell growth (GO:0016049) | 5.75 | 8.21E−04 | 1.54E−02 |
| DNA metabolic process (GO:0006259) | 4.80 | 1.57E−08 | 3.83E−06 |
| DNA repair (GO:0006281) | 4.50 | 5.51E−04 | 1.34E−02 |
| cell cycle (GO:0007049) | 2.86 | 1.40E−05 | 8.52E−04 |
| nucleobase-containing compound metabolic process (GO:0006139) | 1.85 | 6.70E−06 | 5.45E−04 |
| phosphate-containing compound metabolic process (GO:0006796) | 1.83 | 1.36E−03 | 2.36E−02 |
| nitrogen compound metabolic process (GO:0006807) | 1.82 | 3.13E−05 | 1.53E−03 |
| cellular component organization (GO:0016043) | 1.77 | 7.37E−04 | 1.50E−02 |
| cellular component organization or biogenesis (GO:0071840) | 1.75 | 6.99E−04 | 1.55E−02 |
| primary metabolic process (GO:0044238) | 1.46 | 3.88E−04 | 1.05E−02 |
| metabolic process (GO:0008152) | 1.42 | 1.70E−04 | 5.20E−03 |
| cellular process (GO:0009987) | 1.36 | 3.32E−05 | 1.35E−03 |
| Unclassified (UNCLASSIFIED) | 0.73 | 8.92E−05 | 3.11E−03 |
| GGI | | | |
| chromosome segregation (GO:0007059) | 27.94 | 5.56E−15 | 4.52E−13 |
| meiosis (GO:0007126) | 19.96 | 9.33E−07 | 1.63E−05 |
| mitosis (GO:0007067) | 16.07 | 1.92E−16 | 2.35E−14 |
| DNA replication (GO:0006260) | 15.77 | 3.33E−11 | 1.62E−09 |
| cell proliferation (GO:0008283) | 15.40 | 2.56E−05 | 3.91E−04 |
| regulation of cell cycle (GO:0051726) | 13.31 | 2.11E−10 | 8.56E−09 |
| DNA metabolic process (GO:0006259) | 10.02 | 8.84E−14 | 5.39E−12 |
| cell cycle (GO:0007049) | 9.70 | 4.33E−24 | 1.06E−21 |
| cytokinesis (GO:0000910) | 8.97 | 7.07E−05 | 9.59E−04 |
| DNA repair (GO:0006281) | 7.41 | 1.94E−04 | 2.25E−03 |
| reproduction (GO:0000003) | 5.09 | 1.32E−03 | 1.46E−02 |
| cellular component movement (GO:0006928) | 4.73 | 2.52E−05 | 4.09E−04 |
| chromatin organization (GO:0006325) | 4.53 | 2.34E−03 | 2.48E−02 |
| organelle organization (GO:0006996) | 3.91 | 1.69E−08 | 4.59E−07 |
| phosphate-containing compound metabolic process (GO:0006796) | 3.62 | 1.74E−09 | 6.07E−08 |
| cytoskeleton organization (GO:0007010) | 3.57 | 3.78E−03 | 3.84E−02 |
| cellular component organization (GO:0016043) | 2.84 | 5.35E−07 | 1.09E−05 |
| cellular component organization or biogenesis (GO:0071840) | 2.75 | 5.65E−07 | 1.06E−05 |
| nucleobase-containing compound metabolic process (GO:0006139) | 2.58 | 5.88E−08 | 1.43E−06 |
| nitrogen compound metabolic process (GO:0006807) | 2.21 | 8.39E−05 | 1.02E−03 |
| metabolic process (GO:0008152) | 1.90 | 1.76E−07 | 3.89E−06 |
| primary metabolic process (GO:0044238) | 1.78 | 7.16E−05 | 9.19E−04 |
| cellular process (GO:0009987) | 1.73 | 9.27E−09 | 2.83E−07 |
| Unclassified (UNCLASSIFIED) | 0.59 | 5.76E−05 | 8.27E−04 |
| cell surface receptor signaling pathway (GO:0007166) | <0.01 | 4.40E−03 | 4.29E−02 |
| PAM50 | | | |
| meiosis (GO:0007126) | 18.85 | 6.19E−04 | 3.78E−02 |
| negative regulation of apoptotic process (GO:0043066) | 15.74 | 1.40E−04 | 1.14E−02 |
| mitosis (GO:0007067) | 11.81 | 2.40E−06 | 2.93E−04 |
| cell cycle (GO:0007049) | 6.47 | 2.62E−07 | 6.39E−05 |
| TAMR13 | | | |
| chromosome segregation (GO:0007059) | 15.47 | 6.80E−11 | 4.15E−09 |
| meiosis (GO:0007126) | 13.97 | 1.33E−06 | 3.25E−05 |
| cell proliferation (GO:0008283) | 12.93 | 2.15E−06 | 4.37E−05 |
| mitosis (GO:0007067) | 10.18 | 1.63E−13 | 1.98E−11 |
| regulation of cell cycle (GO:0051726) | 9.98 | 8.44E−11 | 4.12E−09 |
| DNA recombination (GO:0006310) | 9.52 | 1.06E−03 | 1.12E−02 |
| regulation of gene expression, epigenetic (GO:0040029) | 6.97 | 3.11E−03 | 3.04E−02 |
| DNA metabolic process (GO:0006259) | 6.65 | 1.80E−11 | 1.46E−09 |
| DNA replication (GO:0006260) | 6.31 | 5.70E−05 | 7.72E−04 |
| cytokinesis (GO:0000910) | 6.28 | 1.71E−04 | 2.09E−03 |
| DNA repair (GO:0006281) | 5.93 | 8.61E−05 | 1.11E−03 |
| cell cycle (GO:0007049) | 5.65 | 1.36E−15 | 3.31E−13 |
| reproduction (GO:0000003) | 4.58 | 2.05E−04 | 2.38E−03 |
| cytoskeleton organization (GO:0007010) | 3.37 | 5.40E−04 | 5.99E−03 |
| cellular component movement (GO:0006928) | 2.84 | 2.08E−03 | 2.12E−02 |
| organelle organization (GO:0006996) | 2.76 | 2.01E−06 | 4.47E−05 |
| phosphate-containing compound metabolic process (GO:0006796) | 2.72 | 6.55E−08 | 1.78E−06 |
| nitrogen compound metabolic process (GO:0006807) | 2.45 | 1.51E−09 | 6.13E−08 |
| biosynthetic process (GO:0009058) | 2.34 | 4.61E−06 | 8.66E−05 |
| cellular component organization (GO:0016043) | 2.14 | 2.73E−05 | 4.44E−04 |
| cellular component organization or biogenesis (GO:0071840) | 2.06 | 4.92E−05 | 7.06E−04 |
| nucleobase-containing compound metabolic process (GO:0006139) | 1.99 | 6.74E−06 | 1.17E−04 |
| metabolic process (GO:0008152) | 1.73 | 2.49E−08 | 7.61E−07 |
| cellular process (G0:0009987) | 1.56 | 1.29E−08 | 4.50E−07 |
| Unclassified (UNCLASSIFIED) | 0.67 | 2.76E−05 | 4.20E−04 |
| Toronto 2017 | | | |
| meiosis (GO:0007126) | 20.16 | 8.81E−07 | 3.58E−05 |
| cell proliferation (GO:0008283) | 18.66 | 1.34E−06 | 4.68E−05 |
| chromosome segregation (GO:0007059) | 15.19 | 6.26E−07 | 3.06E−05 |
| mitosis (GO:0007067) | 13.53 | 7.73E−13 | 9.43E−11 |
| regulation of cell cycle (GO:0051726) | 11.20 | 3.44E−08 | 2.80E−06 |
| amino acid transport (GO:0006865) | 10.04 | 8.06E−04 | 1.16E−02 |
| DNA replication (GO:0006260) | 9.29 | 1.39E−05 | 3.76E−04 |
| cytokinesis (GO:0000910) | 9.06 | 6.70E−05 | 1.36E−03 |
| negative regulation of apoptotic process (GO:0043066) | 8.42 | 1.51E−03 | 1.94E−02 |
| cell cycle (GO:0007049) | 7.20 | 8.96E−15 | 2.19E−12 |
| reproduction (GO:0000003) | 5.14 | 1.25E−03 | 1.70E−02 |
| DNA metabolic process (GO:0006259) | 4.80 | 1.28E−04 | 2.23E−03 |
| cytoskeleton organization (GO:0007010) | 4.64 | 1.62E−04 | 2.48E−03 |
| regulation of phosphate metabolic process (GO:0019220) | 4.27 | 6.21E−05 | 1.38E−03 |
| organelle organization (GO:0006996) | 3.27 | 5.08E−06 | 1.55E−04 |
| phosphate-containing compound metabolic process (GO:0006796) | 2.61 | 6.77E−05 | 1.27E−03 |
| cellular component organization (GO:0016043) | 2.44 | 4.97E−05 | 1.21E−03 |
| cellular component organization or biogenesis (GO:0071840) | 2.28 | 1.58E−04 | 2.57E−03 |
| biological regulation (GO:0065007) | 1.81 | 2.33E−03 | 2.84E−02 |
| cellular process (GO:0009987) | 1.64 | 5.52E−07 | 3.37E−05 |
| Unclassified (UNCLASSIFIED) | 0.69 | 3.59E−03 | 4.18E−02 |

What is claimed is:

1. A method of treating breast cancer in a subject, comprising:
a) obtaining or having obtained an expression level in a sample from a subject for a plurality of genes, wherein the plurality of genes are selected from Table 2;
b) determining that the subject is at low risk of cancer recurrence based on the expression level, or determining that the subject is not at low risk of cancer recurrence based on the expression level; and
c) administering adjuvant chemotherapy or endocrine therapy if the subject is not identified as being at low risk of cancer recurrence based on the expression level, and not administering adjuvant chemotherapy or endocrine therapy if the subject is identified as being at low risk of cancer recurrence based on the expression level.

2. The method of claim 1, further comprising calculating an average genomic risk (AGR) score for the subject, wherein adjuvant chemotherapy or endocrine therapy is administered to the subject if the subject is not identified as being at low risk of cancer recurrence based on both the AGR score and the expression levels of the genes selected from Table 2 in the biological sample, and adjuvant chemotherapy or endocrine therapy is not administered to the subject if the subject is identified as being at low risk of cancer recurrence based on both the AGR score and the expression levels of the genes selected from Table 2 in the biological sample.

3. The method of claim 1, wherein the biological sample is a biopsy or a tumor sample.

4. The method of claim 1, wherein the subject is a human being.

5. The method of claim 1, wherein the subject has estrogen receptor positive (ER+), human epidermal growth factor receptor 2 negative (HER2−) breast cancer and is post-menopausal.

6. The method of claim 1, wherein the expression levels of all of the genes selected from Table 2 are measured in the biological sample.

7. The method of claim 1, wherein the levels of expression of one or more of the genes selected from Table 2 are increased or reduced compared to a control.

8. The method of claim 1, wherein said measuring the levels of expression comprises performing in situ hybridization, a PCR-based method, an array-based method, an immunohistochemical method, an RNA assay method, or an immunoassay method.

9. The method of claim 8, wherein said measuring the levels of expression comprises using a reagent selected from the group consisting of a nucleic acid probe, one or more nucleic acid primers, and an antibody.

10. The method of claim 1, wherein said measuring the level of expression comprises measuring the level of an RNA transcript.

11. The method of claim 1, wherein the method is performed prior to treatment of the subject with the adjuvant chemotherapy or the endocrine therapy.

12. A kit for determining a prognosis of a subject having breast cancer and whether or not to treat the subject with adjuvant chemotherapy and endocrine therapy, the kit comprising agents for measuring levels of expression of a plurality of genes selected from Table 2.

13. The kit of claim 12, wherein the kit comprises agents for measuring the levels of expression of all of the genes listed in Table 2.

14. The kit of claim 12, wherein said agents comprise reagents for performing in situ hybridization, a PCR-based method, an array-based method, an immunohistochemical method, an RNA assay method, or an immunoassay method.

15. The kit of claim 12, wherein said agents comprise one or more of a microarray, a nucleic acid probe, a nucleic acid primer, or an antibody.

16. The kit of claim 12, wherein the kit comprises at least one set of PCR primers capable of amplifying a nucleic acid comprising a sequence of a gene selected from Table 2 or its complement.

17. The kit of claim 12, wherein the kit comprises at least one probe capable of hybridizing to a nucleic acid comprising a sequence of a gene selected from Table 2 or its complement.

18. The kit of claim 12, further comprising information, in electronic or paper form, comprising instructions on how to determine the prognosis of a subject having breast cancer and whether or not to treat the subject with adjuvant chemotherapy and endocrine therapy.

19. The kit of claim 12, further comprising one or more control reference samples.

20. A probe set for determining a prognosis of a subject having breast cancer and whether or not to treat the subject with adjuvant chemotherapy and endocrine therapy, the probe set comprising a plurality of probes for detecting a plurality of target nucleic acids, wherein the plurality of target nucleic acids comprises one or more gene sequences, or complements thereof, of genes selected from Table 2.

21. The probe set of claim 20, wherein the probe set comprises a plurality of probes for detecting a plurality of target nucleic acids comprising gene sequences, or complements thereof, of the genes selected from Table 2.

22. The probe set of claim 21, wherein at least one probe is detectably labeled.

23. A kit for determining a prognosis of a subject having breast cancer and whether or not to treat the subject with adjuvant chemotherapy and endocrine therapy, the kit comprising the probe set of claim 22.

24. A system for determining a prognosis of a subject who has breast cancer and whether or not to treat the subject with adjuvant chemotherapy and endocrine therapy, the system comprising:
a) the probe set of claim 22; and
b) a computer model or algorithm for analyzing an expression level or expression profile of the plurality of target nucleic acids hybridized to the plurality of probes in a biological sample from the subject who has breast cancer and determining if the subject is at low risk of cancer recurrence based on the expression level or expression profile and can be spared treatment with adjuvant chemotherapy and endocrine therapy.

25. A kit comprising the system of claim 24.

26. The kit of claim 25, further comprising a computer model or algorithm for designating a treatment modality for the subject.

27. The kit of claim 25, further comprising a computer model or algorithm for normalizing the expression level or expression profile of the plurality of target nucleic acids.
